(19) United States
(12) Patent Application Publication
(10) Pub. No.: US 2026/0240850 A1
(43) Pub. Date: Aug. 20, 2026

(54) Compositions and Methods for Treating MALT1 Associated Diseases

(71) Applicant: Rarefied Biosciences, Inc., Santa Monica (CA)

(72) Inventor: Manish Butte, Santa Monica, CA (US)

(73) Assignee: Rarefied Biosciences, Inc., Santa Monica, CA (US)

(21) Appl. No.: 19/159,477

(22) PCT Filed: Feb. 29, 2024
(86) PCT No.: PCT/US2024/017855

§ 371 Date: Aug. 25, 2025

Related U.S. Application Data
(60) Provisional application No. 63/487,833, filed on Mar. 1, 2023.

(51) Int. Cl. A61K 31/519; A61K 31/415

(52) U.S. Cl. CPC A61K 31/519; A61K 31/415

(57) ABSTRACT

The present disclosure provides MALT1 inhibitors for reducing signaling by Jun/Fos, and/or NF-κB, and/or a mTor pathway (e.g., mTORc1 and/or mTORc2) activation in cells that have gain of function alleles that act through MALT1. The disclosure also provides for the treatment of patients with these gain of function alleles by the administration of MALT1 inhibitors. In an application, the MALT1 inhibitors are administered to a patient with BENTA.

### BACKGROUND

BENTA, B-cell expansion with NF-κB and T-cell anergy, is a rare genetic disorder of the immune system caused by mutations in the gene CARD11 (Caspase recruitment domain family member 11). The disease is characterized by high levels of certain B-cells starting in infancy (B-cell lymphocytosis), an enlarged spleen (splenomegaly), enlarged lymph nodes (lymphadenopathy), immunodeficiency, and an increased risk of lymphoma.

Currently, minimal treatment options are available for people with BENTA disease. Treatment options mostly address the infections associated with BENTA and/or the B-cell cancers associated with BENTA. There is a need for new medical treatments that address the underlying causes of BENTA as a cure, rather than mere treatment of the symptoms from BENAT. It is an object of this disclosure to provide methods of treating the underlying genetic cause of BENTA.

### SUMMARY

The disclosure relates to the use of MALT1 inhibitors to reduce activation of Jun/Fos, and/or NF-κB, and/or mTor pathways (e.g., mTORc1 and/or mTORc2) resulting from gain of function mutations that act through MALT1 (e.g., CARD11 or CARD14 gain of function mutants). Such gain of function mutations cause diseases such as B-cell expansion with NF-κB and T-cell anergy (BENTA), A20 haploinsufficiency, CARD14 generalized pustular psoriasis, HOIL1 hypomorphism, and NF-kB gain of function syndromes. MALT1 inhibitors include, for example, Compounds 1.1, 1.26, 1.27, 1.30, 1.33, and/or 2.4. MALT1 inhibitors also include compounds of Formula 1:

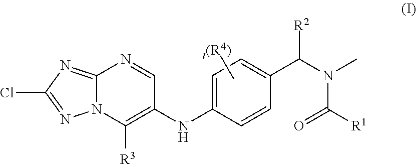

or a pharmaceutically acceptable salt thereof, wherein:
R¹ is selected from the group consisting of C1-6 alkyl, C1-6 alkoxy, C3-6 cycloalkyl and 5-10 membered heterocyclyl, wherein the C1-6 alkyl, C3-6 cycloalkyl, and 5-10 membered heterocyclyl may be optionally substituted on one or more available carbons by one, two, three, or more substituents each independently selected from Ria, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH3 or CF3;
R3 is hydrogen; or
R3 is selected from the group consisting of C1-6 alkyl, C1-6 alkoxy, C3-7 cycloalkyl, 5-6 membered heterocyclyl, 5-6 membered heterocyclyl-C1-3 alkyl 5-6 membered heterocyclyl-O—, phenyl, and 5-6 membered heteroaryl, any of which may be optionally substituted with one, two or three substituents each independently selected from R^{3a};
R4 is C1-6 alkyl;
R^{1a} is independently, for each occurrence, selected from the group consisting of cyano, halogen, hydroxyl, oxo, C₁₋₆ alkyl, —C(O)OR^{A}, —C(O)N(R^{A})₂, —N(R^{A})₂, C1-6 alkoxy, 5-6 membered heterocyclyl, and 5-6 membered heteroaryl, wherein the C1-6 alkyl is optionally substituted with N(R^{A})₂, and wherein if the 5-6 membered heterocyclyl contains a substitutable ring nitrogen atom, that ring nitrogen atom may optionally be substituted by R^{p};
R^{1b} is selected from the group consisting of C1-6 alkyl, —C(O)OR^{A}, —C(O)C1-6 alkyl, —C(O)C3-6 cycloalkyl, —C(O)N(R^{A})₂, and —S(O)₂C1-6 alkyl;
R^{3a} is independently, for each occurrence, selected from the group consisting of halogen, C1-4 alkyl, C1-4 haloalkyl, C1-4 alkoxy, C1-4 haloalkoxy, hydroxy, C1-4 alkenyl, cyano, azido, —NR^{C}R^{D}, C3-6 cycloalkyl, Ci1-4 alkoxy, 5-6 membered heterocyclyl-O—, 5-6 membered heterocyclyl, and phenyl, wherein C3-6cycloalkyl, 5-6 membered heterocyclyl-O—, 5-6 membered heterocyclyl, and phenyl are optionally substituted with one, two or three substituents each independently selected from R^{p};
R^{p} is independently, for each occurrence, selected from the group consisting of halogen, C1-4 alkyl, C1-4 haloalkyl, hydroxy, C1-4 alkoxy, C1-4 alkoxyC1-4 alkyl, NR^{C}R^{D}, and aminoC1-3 alkyl;
R^{A} is independently, for each occurrence, selected from the group consisting of hydrogen, C1-6 alkyl, —C(O)C1-6 alkyl, and —C(O)OC1-6 alkyl;
R^{B} is selected from the group consisting of Ci1-6 alkyl, C1-6 cycloalkyl, and —C(O)OC1-6 alkyl;
R^{C} and R^{D} are independently, for each occurrence, selected from the group consisting of hydrogen, C1-6 alkyl, haloC1-6 alkyl, and C-3-4 cycloalkyl, or
R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and
T is 0 or 1.

### BRIEF DESCRIPTION OF THE FIGURES

FIG. 1 shows a diagram of a pathway for MALT1 signal transduction to NF-κB.

FIG. 2 shows a diagram of a pathway for MALT1 signal transduction to NF-κB with CARD11 gain of function alleles. This drawing also shows inhibition of CARD11 stimulation by a MALT1 inhibitor.

FIG. 3 shows a diagram of a pathway for MALT1 signal transduction to NF-κB with CARD14 gain of function alleles. This drawing also shows inhibition of CARD14 stimulation by a MALT1 inhibitor.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a” “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modem Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et at., Enantiomers, Racemates and Resolutions (Wiley Interscience, New York, 1981); Wilen et at., Tetrahedron 33:2725 (1977); Eliel, Stereochemistry of Carbon Compounds (McGraw-Hill, NY, 1962); and Wilen, Tables of Resolving Agents and Optical Resolutions p. 2.68 (E,L, Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, the term “pure enantiomeric compound” is substantially free from other enantiomers or stereoisomers of the compound (i.e., in enantiomeric excess), in other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In an aspect, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain aspects, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain aspects, the enantiomerically pure S-compound in such compositions can, for example, comprise, at least about 95% by weight S-compound and at most about 5% by weight R-compound, by total weight of the compound. In certain aspects, the active ingredient can be formulated with little or no excipient or carrier.

Compound described herein may also comprise one or more isotopic substitutions. For example, H may be in any isotopic form, including ′H, H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁵F and ¹⁹F; and the like.

When a range of values is listed, it is intended to encompass each value and sub-range within the range. For example, “Ci-6 alkyl” is intended to encompass, Ci, Cu, Cs, Cr, Cs, O, Ci-e, Ci-5, Ci-4, Ci-3, Ci-2, C2-6, C2-5, C2-4, C2-3, C3-6, C3-5, C3-4, C4-6, C4-5, and C5-6 alkyl.

As used herein, the term “alkyl” refers to a radical of a straight-chain or branched saturated hydrocarbon group, e.g., having 1 to 20 carbon atoms (“C1-20 alkyl”). In some aspects, an alkyl group has 1 to 10 carbon atoms (“C1-10 alkyl”). In some aspects, an alkyl group has 1 to 9 carbon atoms (“Ci-9 alkyl”). In some aspects, an alkyl group has 1 to 8 carbon atoms (“Ci-8 alkyl”). In some aspects, an alkyl group has 1 to 7 carbon atoms (“Ci-7 alkyl”). In some aspects, an alkyl group has 1 to 6 carbon atoms (“Ci-6 alkyl”). In some aspects, an alkyl group has 1 to 5 carbon atoms (“Ci-5 alkyl”). In some aspects, an alkyl group has 1 to 4 carbon atoms (“C1-4 alkyl”). In some aspects, an alkyl group has 1 to 3 carbon atoms (“C1-3 alkyl”). In some aspects, an alkyl group has 1 to 2 carbon atoms (“C1-2 alkyl”). In some aspects, an alkyl group has 1 carbon atom (“C1 alkyl”). Examples of Ci-6 alkyl groups include methyl, ethyl, propyl, isopropyl, butyl, isobutyl, pentyl, hexyl, and the like.

As used herein, the term “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C2-20 alkenyl”). In certain aspects, alkenyl does not contain any triple bonds. In some aspects, an alkenyl group has 2 to 10 carbon atoms (“C2-10 alkenyl”). In some aspects, an alkenyl group has 2 to 9 carbon atoms (“C2-9 alkenyl”). In some aspects, an alkenyl group has 2 to 8 carbon atoms (“C2-8 alkenyl”). In some aspects, an alkenyl group has 2 to 7 carbon atoms (“C2-7 alkenyl”). In some aspects, an alkenyl group has 2 to 6 carbon atoms (“C2-6 alkenyl”). In some aspects, an alkenyl group has 2 to 5 carbon atoms (“C2-5 alkenyl”). In some aspects, an alkenyl group has 2 to 4 carbon atoms (“C2-4 alkenyl”). In some aspects, an alkenyl group has 2 to 3 carbon atoms (“C2-3 alkenyl”). In some aspects, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (Ce), and the like. Additional examples of alkenyl include heptenyl (C7), octenyl (C8), octatrienyl (C8), and the like.

As used herein, the term “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C2-20 alkynyl”). In certain aspects, alkynyl does not contain any double bonds. In some aspects, an alkynyl group has 2 to 10 carbon atoms (“C2-10 alkynyl”). In some aspects, an alkynyl group has 2 to 9 carbon atoms (“C2-9 alkynyl”). In some aspects, an alkynyl group has 2 to 8 carbon atoms (“C2-8 alkynyl”). In some aspects, an alkynyl group has 2 to 7 carbon atoms (“C2-7 alkynyl”). In some aspects, an alkynyl group has 2 to 6 carbon atoms (“C2-6 alkynyl”). In some aspects, an alkynyl group has 2 to 5 carbon atoms (“C2-5 alkynyl”). In some aspects, an alkynyl group has 2 to 4 carbon atoms (“C2-4 alkynyl”). In some aspects, an alkynyl group has 2 to 3 carbon atoms (“C2-3 alkynyl”). In some aspects, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like.

As used herein, the term “alkylene,” “alkenylene,” “alkynylene,” “cycloalkylene,” “heterocyclylene,” “heteroarylene,” and “phenylene” refer to a divalent radical of an alkyl, alkenyl, alkynyl, cycloalkyl, heterocyclyl (e g., saturated and partially saturated), heteroaxyl, and phenyl group respectively. When a range or number of carbons is provided for a particular “alkylene,” “alkenylene,” or “alkynylene,” group, it is understood that the range or number refers to the range or number of carbons entire linear carbon chain. “Alkylene,” “alkenylene,” and “alkynylene,” groups may be substituted or unsubstituted with one or more substituents as described herein.

As used herein, the term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). In some aspects, an aryl group has six ring carbon atoms (“C6 aryl”; e.g., phenyl). In some aspects, an aryl group has ten ring carbon atoms (“C10 aryl”; e.g., naphthyl such as 1-naphthyl and 2-naphthyl). In some aspects, an aryl group has fourteen ring carbon atoms (“C14 aryl”). “Aryl” also includes ring systems wherein the aryl ring, as defined above, is fused with one or more carbocyclic or heterocyclic groups wherein the radical or point of attachment is on the aryl ring, and in such instances, the number of carbon atoms continue to designate the number of carbon atoms in the aryl ring system.

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexaiene, as-indacene, s-indacene, indane, indene, naphthalene, octacene, octaphene, octaiene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

As used herein, the term “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclic or heterocyclic groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system, Bicyclic heteroaryl groups wherein one ring does not contain a heteroatom (e.g., indoyl, quinolyl, carbazolyl, and the like) the point of attachment can be on either ring, i.e., either the ring bearing a heteroatom (e.g., 2-indolyi) or the ring that does not contain a heteroatom (e.g., 5-indolyl).

In some aspects, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some aspects, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some aspects, a heteroaryl group is a 5-6 membered aromatic ring system having ring carbon atoms and 1-4 ring heteroatoms, wherein each heteroatom is independently selected from nitrogen, oxygen, and sulfur (“5-6 membered heteroaryl”). In some aspects, the 5-6 membered heteroaryl has 1-3 ring heteroatoms selected from nitrogen, oxygen, and sulfur. In some aspects, the 5-6 membered heteroaryl has 1-2 ring heteroatoms selected from nitrogen, oxygen, and sulfur. In some aspects, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazoiyl, benzotriazolyl, benzothiophenyl, isobenzothiophenyl, benzofuranyl, benzoisofuranyl, benzimidazolyl, benzoxazolyl, benzisoxazoiyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Examples of representative heteroaryls include the following:

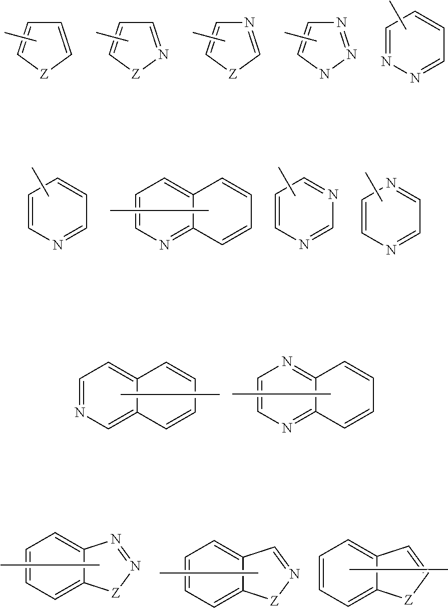

wherein each Z is selected from carbonyl, N, NR65, O, and 8; and R65 is independently hydrogen, Ci-8 alkyl, C3-10 carbocyclyl, 4-10 membered heterocyclyl, C6-C10 aryl, and 5-10 membered heteroaryl.

As used herein, the term “carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system, in some aspects, a carbocyclyl group has 3 to 8 ring carbon atoms (“C3-8 carbocyclyl”). In some aspects, a carbocyclyl group has 3 to 7 ring carbon atoms (“C3-7 carbocyclyl”). In some aspects, a carbocyclyl group has 3 to 6 ring carbon atoms (“C3-6 carbocyclyl). In some aspects, a carbocyclyl group has 5 to 10 ring carbon atoms (“C5-10 carbocyclyl”). Exemplary C3-6 carbocyclyl groups include, without limitation, cyclopropyl (C3),cyclobutyl (C₄), cyclobutenyl (C4), cyclopentyl (C5), cyclopentenyl (C5), cyclohexyl (C6), cyclohexenyl (G6), cyclohexadienyl (G6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo-octanyl (C8), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or contain a fused, bridged or spiro ring system such as a bicyclic system (“bicylic carbocyclyl”) and can be saturated or can be partially unsaturated. “Carbocyclic’ also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

As used herein, the term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “Cycloalkyl,” derived from a cycloalkane. Exemplary cycloalkyl groups include, but are not limited to, cyclohexanes, cyclopentanes, cyclobutanes and cyclopropanes.

As used herein, the term “C3-6 monocyclic cycloalkyl” or “monocyclic C3-6 cycloalkyl” refers to a 3- to 7-membered monocyclic hydrocarbon ring system that is saturated. 3- to 7-membered monocyclic cycloalkyl groups include, without limitation, cyclopropyl, cyclobutyl, cyclopentyl, and cyclohexyl. Where specified as being optionally substituted or substituted, substituents on a cycloalkyl (e.g., in the ease of an optionally substituted cycloalkyl) may be present on any substitutable position and, include, e.g., the position at which the cycloalkyl group is attached.

As used herein, the term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). Heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.
in some embodiments, a heterocyclic group is a 4-7 membered non-aromatic ring system having ring carbon atoms and 1-4 ring heteroatoms, wherein each heteroatom is independently selected from nitrogen, oxygen, and sulfur (“4-7 membered heterocyclyl”). In some aspects, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”).

In some aspects, a heterocyclyl group is a 5-8 membered non-aromatic ring system having ring carbon atoms and 1-4 ring heteroatoms, wherein each heteroatom is independently selected from nitrogen, oxygen, and sulfur (“3-8 membered heterocyclyl”). In some aspects, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some aspects, the 5-6 membered heterocyclyl has 1-3 ring heteroatoms selected from nitrogen, oxygen, and sulfur. In some aspects, the 5-6 membered heterocyclyl has 1-2 ring heteroatoms selected from nitrogen, oxygen, and sulfur. In some aspects, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary7 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrroiyl and pyrrolyi-2,5-dione. Exemplary′ 5-membered heterocyclyl groups containing two heteroatoms include, without limitation, dioxolanyl, oxasulfuranyl, disulfuranyl, and oxazolidin-2-one. Exemplary′ 5-membered heterocyclyl groups containing three heteroatoms include, without limitation, triazolinyl, oxadiazolinyl, and thiadiazolinyl.

Exemplary′ 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary7 6-membered heterocyclyl groups containing two heteroatoms include, without limitation, triazinanyl. Exemplary 7-membered heterocyclyl groups containing one heteroatom include, without limitation, azepanyl, oxepanyl, and thiepanyl. Exemplary 8-membered heterocyclyl groups containing one heteroatom include, without limitation, azocanyl, oxecanyl and thiocanyl. Exemplary 5-membered heterocyclyl groups fused to a C5 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicylic heterocyclic ring) include, without limitation, tetrahydroquinolinyl, tetralrydroisoquinolinyl, and the like.

Examples of saturated or partially unsaturated heterocyclic radicals include, without limitation, tetrahydrofuranyl, tetrahydrothienyl, terahydropyranyl, pyrrolidinyl, pyridinonyl, pyrroiidonyl, piperidinyl, oxazolidinyl, piperazinyl, dioxanyl, dioxolanyl, morphoiinyl, dihydrofuranyl, dihydropyranyl, dihydropyridinyl, tetrahydropyridinyl, dihydropyrimidinyl, oxetanyl, azetidinyl and tetrahydropyrimidinyl. Where specified as being optionally substituted or substituted, substituents on a heterocyclyl (e.g., in the ease of an optionally substituted heterocyclyl) may be present on any substitutable position and, include, e.g., the position at which the heterocyclyl group is attached.

As used herein, the term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

As used herein, the term “cyano” refers to —CN.

As used herein, the term “halo” and “halogen” as used herein refer to an atom selected from fluorine (fluoro, —F), chlorine (chloro, —Cl), bromine (bromo, —Br), and iodine (iodo, —I). In certain aspects, the halo group is either fluoro or chloro.

As used herein, the term

As used herein, the term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, the term “Fluoroalkoxy” is a haloalkyl group which is attached to another moiety via an oxygen atom such as, e.g., but are not limited to —OCHCFH2 or —OCF3.

As used herein, the term “oxo” refers to —C=0.

As used herein, the term “substituted”, whether preceded by the term “optionally” or not, means that at least one hydrogen present on a group (e g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR, —N(R)2, —CN, —C(═O)R, —C(═O)N(R)2, CO—R N SO′R′3, —C(NR)R, —C(—NR)R, —C(—NR)N(R)2, SO.NIR SO.′R SO.—OR″. —SOR, —C(═S)N(RCC)2, —C(═O)SR, C(S)SR—, P(═O)2R, —P(═O)(R)2, —P(═O)2N(R)2, —P(═O)(NR)₂, C1-10 alkyl, C1-10 perhaloalkyl, C2-10 alkenyl, C2-10 alkynyl, C3-10 carbocyclyl, 3-14 membered heterocyclyl, C6-11 aryl, and 5-14 membered heteroaryl, or two Rcc groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 RQQ groups.

As used herein, the term “coding sequence” is defined to mean a portion of a nucleic acid (e.g., a gene) that encodes an amino acid sequence of a protein.

As used herein, the term “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the terms “percentage of sequence identity” and “percentage homology” are used interchangeably and are defined to mean comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Nat Acad Sci. USA 89:10915, 1989). Exemplary determination of sequence alignment and % sequence identity can also employ the BESTFIT or GAP programs in the GCG Wisconsin Software package (Accelrys, Madison WI), using default parameters provided.

As used herein, the term “pharmaceutically acceptable earner” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al, describes pharmaceutically acceptable salts in detail in J Pharmaceutical Sciences (1977) 66:1-19, which is incorporated by reference in its entirety for all purposes. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, algmate, ascorbate, aspartate, benzenesulfonate, benzoate, bisulfate, borate, butyrate, eaniphorate, camphorsulfonate, citrate, cyelopentanepropionate, digluconate, dodecyisulfate, ethanesulfonate, formate, fumarate, glucoheptonate, glycerophosphate, gluconate, hemisulfate, heptanoate, hexanoate, hydroiodide, 2-hydroxy-ethanesullbnate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(Ci-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “reference sequence” is defined to mean a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In an aspect, a “reference sequence” can be based on a primary amino acid sequence, where the reference sequence is a sequence that can have one or more changes to the primary sequence.

As used herein, the term “substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

As used herein, the term a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “amino acid substitution” or “amino acid difference” are defined to mean a change in the amino acid residue at a position of a polypeptide sequence relative to the amino acid residue at a corresponding position in a reference sequence which is the primary translation product starting at the methionine initiation codon. The positions of amino acid differences generally are referred to herein as “Xn,” where n refers to the corresponding position in the reference sequence upon which the residue difference is based. For example, a “residue difference at position X as compared to the primary translation product starting at the methionine initiation codon” refers to a change of the amino acid residue at the polypeptide position corresponding to position X of a wild-type protein. Thus, if the reference polypeptide of the primary translation starting at the methionine initiation codon product for a wild type gene has a valine at position X, then an “amino acid substitution” or “residue difference at position X as compared to reference sequence” refers to an amino acid substitution of any residue other than valine at the position of the polypeptide corresponding to position X of the reference sequence. In most instances herein, the specific amino acid substitution or amino acid residue difference at a position is indicated as “XnY” where “Xn” specifies the corresponding position as described above, and “Y” is the single letter identifier of the amino acid found in the engineered polypeptide (i.e., the different residue than in the reference polypeptide). In an aspect, where more than one amino acid can appear at a specified residue position, the alternative amino acids can be listed in the form XnY/Z, where Y and Z represent alternate amino acid residues. In some instances, the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present disclosure can include one or more amino acid residue differences relative to a reference sequence, which is indicated by a list of the specified positions where changes are made relative to the reference sequence.

As used herein, the terms “conservative amino acid substitution” or “conservative amino acid difference” are defined to mean a change in the amino acid at a residue position to a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively. Exemplary conservative substitutions are provided in Table 1 below.

TABLE 1

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

As used herein, the terms “non-conservative substitution” or “non-conservative amino acid difference” are defined to mean a change in the amino acid at a residue position to a different residue with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, the term “deletion” is defined to mean a modification of a polypeptide by removal of one or more amino acids from the reference polypeptide or modification of a nucleic acid by removal of one or more nucleotides from the reference nucleic acid. For example, deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide. Deletions can be directed to the internal portions and/or terminal portions of the polypeptide. In various embodiments, the deletion can comprise a continuous segment or can be discontinuous.

As used herein, the term “insertion” is defined to mean a modification to a polypeptide by addition of one or more amino acids from the reference polypeptide, or modification of a nucleic acid by addition of one or more nucleic acids. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the reference polypeptide.

As used herein, the term “specificity” as used in reference to an biocatalyst or enzyme is defined to mean the discrimination of the biocatalyst for a substrate compound.

As used herein, the term “relative specificity” is defined to mean the specificity of a biocatalyst or enzyme for one substrate compound over another or other substrate compounds.

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As defined herein, the term “heterologous” polynucleotide or polypeptide is defined to mean any polynucleotide or polypeptide that is not naturally found in a host cell. As such, the term includes polynucleotides that are removed from a host cell, subjected to laboratory manipulation, and then reintroduced into a host cell. In some embodiments, the introduced polynucleotide expresses the heterologous polypeptide.

As used herein, the term “control sequence” is defined to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and where appropriate, translational stop signals. The control sequences may be provided with linkers for the purpose of introducing specific restriction sites facilitating ligation of the control sequences with the coding region of the nucleic acid sequence encoding a polypeptide.

As used herein, the term “operably linked” is defined to mean a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the term “promoter sequence” is defined to mean a nucleic acid sequence that is recognized by a host cell for expression of a polynucleotide of interest, such as a coding sequence or gene. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

### MALT1 Inhibitors

Mucosa-Associated Lymphoid Tissue Lymphoma Translocation Protein 1 (MALT1). MALT1 is a caspase-like protease that plays a role in BCL10-induced activation of NF-κB, and/or activation of mTOR pathway signaling (e.g., mTORc1 and/or mTORc2), and/or activation of Jun/Fos. The protein can be a component of the CARMA1-BCL10-MALT1 (CBM) signalosome, and other signalosomes (e.g., involving CARD11 or CARD14) that triggers NF-κB signaling, mTor pathway signaling (e.g., mTORc1 and/or mTORc2), and Jun/Fos signaling leading to lymphocyte activation following antigen-receptor stimulation. Biallelic loss of function mutations in this gene result in immunodeficiency 12 (IMD12).

MALT1 inhibitors include compounds of Formula I:

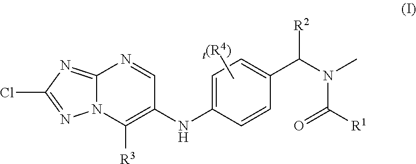

or a pharmaceutically acceptable salt thereof, wherein:
R¹ is selected from the group consisting of C1-6 alkyl, C1-6 alkoxy, C3-6 cycloalkyl and 5-10 membered heterocyclyl, wherein the C1-6 alkyl, C3-6 cycloalkyl, and 5-10 membered heterocyclyl may be optionally substituted on one or more available carbons by one, two, three, or more substituents each independently selected from Ria, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH3 or CF3;
R3 is hydrogen; or
R3 is selected from the group consisting of C1-6 alkyl, C1-6 alkoxy, C3-7 cycloalkyl, 5-6 membered heterocyclyl, 5-6 membered heterocyclyl-C1-3 alkyl 5-6 membered heterocyclyl-O—, phenyl, and 5-6 membered heteroaryl, any of which may be optionally substituted with one, two or three substituents each independently selected from R^{3a};
R4 is C1-6 alkyl;
R^{1a} is independently, for each occurrence, selected from the group consisting of cyano, halogen, hydroxyl, oxo, C₁₋₆ alkyl, —C(O)OR^{A}, —C(O)N(R^{A})₂, —N(R^{A})₂, C1-6 alkoxy, 5-6 membered heterocyclyl, and 5-6 membered heteroaryl, wherein the C1-6 alkyl is optionally substituted with N(R^{A})₂, and wherein if the 5-6 membered heterocyclyl contains a substitutable ring nitrogen atom, that ring nitrogen atom may optionally be substituted by R^{p};
R^{1b} is selected from the group consisting of C1-6 alkyl, —C(O)OR^{A}, —C(O)C1-6 alkyl, —C(O)C3-6 cycloalkyl, —C(O)N(R^{A})₂, and —S(O)₂C1-6 alkyl;
R^{3a} is independently, for each occurrence, selected from the group consisting of halogen, C1-4 alkyl, C1-4 haloalkyl, C1-4 alkoxy, C1-4 haloalkoxy, hydroxy, C1-4 alkenyl, cyano, azido, —NR^{C}R^{D}, C3-6 cycloalkyl, Ci1-4 alkoxy, 5-6 membered heterocyclyl-O—, 5-6 membered heterocyclyl, and phenyl, wherein C3-6cycloalkyl, 5-6 membered heterocyclyl-O—, 5-6 membered heterocyclyl, and phenyl are optionally substituted with one, two or three substituents each independently selected from R^{p};
R^{p} is independently, for each occurrence, selected from the group consisting of halogen, C1-4 alkyl, C1-4 haloalkyl, hydroxy, C1-4 alkoxy, C1-4 alkoxyC1-4 alkyl, NR^{C}R^{D}, and aminoC1-3 alkyl;
R^{A} is independently, for each occurrence, selected from the group consisting of hydrogen, C1-6 alkyl, —C(O)C1-6 alkyl, and —C(O)OC1-6 alkyl;
R^{B} is selected from the group consisting of Ci1-6 alkyl, C1-6 cycloalkyl, and —C(O)OC1-6 alkyl;
R^{C} and R^{D} are independently, for each occurrence, selected from the group consisting of hydrogen, C1-6 alkyl, haloC1-6 alkyl, and C-3-4 cycloalkyl, or
R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and
T is 0 or 1.

MALT1 inhibitors also include, for example,

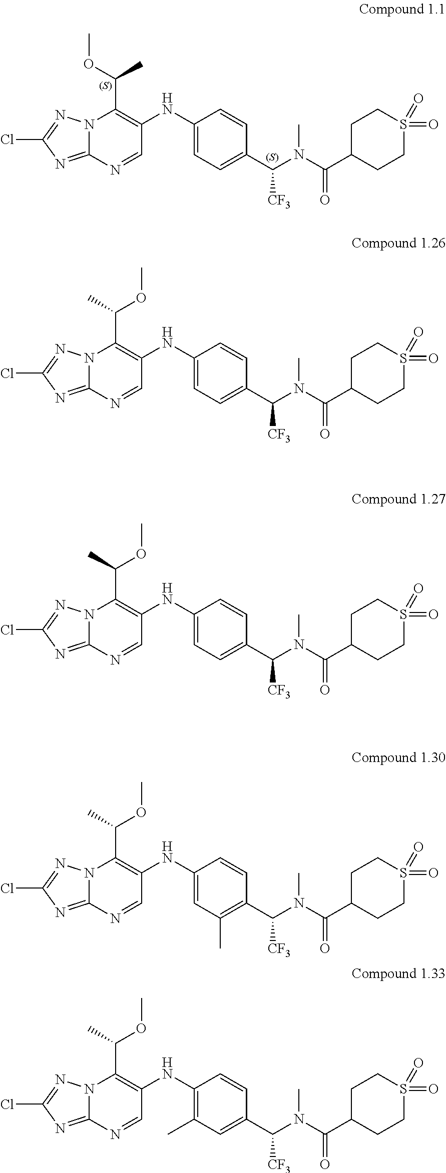

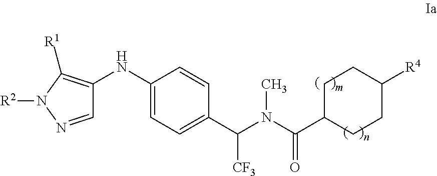

or a stereoisomer and/or a pharmaceutically acceptable salt thereof, wherein:
R¹ is C₁₋₆alkyl or C₁₋₃haloalkyl, wherein the C₁₋₆alkyl may be optionally substituted with —O—C₁₋₃alkyl;
R² is aryl or 5-6 membered heteroaryl, wherein the aryl may be optionally substituted with cyano;
R⁴ is —C(O)OH or 5-6 membered heteroaryl;
m is 0 or 1; and
n is 0 or 1.

Other MALT1 inhibitors include, for example:

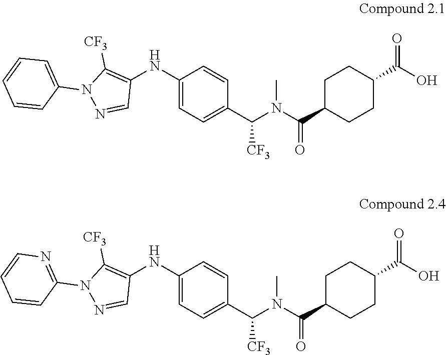 [SMILES: CN(C(=O)[C@H]1CC[C@H](C(=O)O)CC1)[C@@H](c1ccc(Nc2cnn(-c3ccccc3)c2C(F)(F)F)cc1)C(F)(F)F ; CN(C(=O)[C@H]1CC[C@H](C(=O)O)CC1)[C@@H](c1ccc(Nc2cnn(-c3ccccn3)c2C(F)(F)F)cc1)C(F)(F)F]

The MALT1 inhibitors include pharmaceutically acceptable salts, pharmaceutically acceptable esters, tautomeric forms, polymorphs, and prodrugs of such compounds. In some aspects, the MALT1 inhibitors include a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture), a pure or enriched stereoisomer of a compound described herein, e.g. a compound of Formula I); such as a compound of Formula named herein.

### Synthesis of MALT1 Inhibitors

A mixture of tert-butyl N-{2-chloro-7-[(1S)-1-methoxyethyl]-[1,2,4]triazolo[1,5-a]pyfimidin-6-yl}carbamate [INT 1-e](420 mg, 1.28 mmol) in 4N HCl/dioxane (5 mL) was stirred at 2.5° C. for 2 h. LCMS showed the reaction was completed and one new peak with desired MS was detected (Rt=0.611 min, m/z: 227.8 [M+H]+). The mixture was concentrated under reduced pressure to afford 2-chloro-7-[(1S)-1-methoxyethyl]-[1,2,4]triazolo[1,5-a]pyrimidin-6-amine hydrochloride [INT 1.1](400 mg, crude) as a solid.

(R)-2-chloro-7-(1-methoxyethyl)-[1,2,4]triazolo[1,5-a]pyrimidin-6-amine [INT 1.3] can be prepared by the same synthetic route as outlined for INT 1.1 using (2R)-2-methoxypropanoic acid as a starting material, m/z: [M+H]+ Calcd for C8H11C1N50 228.1; Found 228.1.

To a solution of 4-bromobenzaldehyde [INT 2-a](100 g, 541 mmol, 1.0 eq) m toluene (500 mL) was added (R)-2-methylpropane-2-sulfinamide (72.1 g, 595 mmol, 1.5 eq) at 25° C. The mixture was stirred at 25° C. for 15 mins. Then to above reaction was added NaOH (21.6 g, 541 mmol, 1.0 eq) and the mixture was stirred at 2.5° C. for 12 h. Na₂SO₄ (50 g) was added to the mixture and stirred for 20 mins. Four reaction mixtures were combined and filtered through celite to give the filtrate which was concentrated in vacuum to give the crude product as an oil. The crude product was dissolved in Petroleum ether (1.0 L) and stirred at −50° C. for 1.0 h, filtered to give (R,E)-N-(4-brornobenzylidene)-2-methylpropane-2-sulfinamide [INT 2-b](620 g, 2.15 mmol, 99.5% yield) as a solid.

To a solution of (R,E)-N-(4-bromobenzylidene)-2-metlxylpropane-2-sulfinamide [INT 2-b](2.06 g, 715 mmol, 1.0 eq) and tetrabutylammonium acetate (216 g, 715 mmol, 21.8 mL, 1.0 eq) in DMF (1.4 L) was added TMSCF₃ (259 g, 1.82 mol, 2.5 eq) at 0° C. The mixture was stirred at 5° C. for 1.5 h. This process was repeated 2 times and the three reaction mixtures were combined for work-up. The mixture was poured into saturated NH₄Cl solution (1.3.0 L) and stirred for 10 mins to give the suspension. The suspension was filtered to give the filter cake and eluted with water (5.0 L). The filter cake was triturated with MTBE/Petroleum ether (v/v=1:4, 2.0 L) and to give the product as a solid and the mother liquid was concentrated in vacuum to give the crude product as an oil which was purified by column chromatography on silica gel with petroleum ether/ethyl acetate (10/1-1/1) to give (R)—N—((S)-1-(4-bromophenyl)-2,2,2-trifluoroethyl)-2-methyipropane-2-sulfinamide [INT 2.1](389 g, 1.09 mol, 50.6% yield) as a solid. ¹HNMR (400 MHz, CDCb) δ=1.25 (s, 9H), 3.64 (d, J=6.40 Hz, 1H), 4.79-4.83 (m, 1H), 7.32 (d, I=8.40 Hz, 2H), 7.56 (d, I=6.40 Hz, 2H).

(S)—N—((R)-1-(4-bromophenyl)-2,2,2-trifluoroethyl)-2-methylpropane-2-sulfinamide [INT 14-a]was prepared by the same synthetic route as outlined for (R)—N—((S)-1-(4-bromophenyl)-2,2,2-trifluoroethyl)-2-methylpropane-2-sulfmamide [INT 2.1] using (S)-(−)-2-methyl-2-propanesulfinamide.

To a mixture of (S)—N—((R)-1-(4-bromophenyl)-2,2,2-trifluoroethyl)-2-methylpropane-2-sulfinamide [INT 14-a](8 g, 22.3 mmol) in MeOH (60 mL) was added 4 M HCl in dioxane (20 mL). The mixture was stirred at 20° C. for 1.5 h. The mixture was concentrated under reduced pressure to afford the crude product. The mixture was diluted with water (50 mL) and extracted with EtOAc (50 mL×2). The combined organic layers were washed with 1 M HCl (50 mL×2). The aqueous phase was basified with 2 N NaOH to pH=9-10 and extracted with CH₂Cl₂ (50 mL×2).

The combined organic layers were dried over anhydrous Na₂SO₄ filtered, and concentrated under reduced pressure to give the crude product (R)-1-(4-bromophenyl)-2,2,2-trifluoroethan-1-amine [INT 14-b](3.50 g, 13.7 mmol, 61.8% yield) as a yellow solid, m/z: [M+H]+ Calcd for C8H8BrF3N, 254.0, 256.0; Found 254.1.

To a mixture of tetrahydro-2H-thiopyran-4-carboxylic acid 1,1-dioxide [INT 4-a](1.68 g, 9.44 mmol), EDCI (2.26 g, 11.8 mmol), and HOBt (1.59 g, 11.8 mmol) in CH2CI2 (20 ml) was added (R)-1-(4-bromophenyl)-2,2,2-trifluoroethan-1-amine [INT 14-b](2 g, 7.87 mmol). The mixture was stirred at 25° C. for 16 hr. The reaction was quenched by adding water (50 mL) and was extracted with EtOAc (50 mL×3). The combined organic layers were washed with brine (50 mL×2), dried over anhydrous Na₂SO₄ and concentrated under reduced pressure to give the crude product, which was purified by flash chromatography on silica gel (EtOAc/PE=0/1 to 1/5) to give (R)—N-(1-(4-bromophenyl)-2,2,2-trifluoroethyl)tetrahydro-2H-thiopyran-4-carboxamide 1,1-dioxide [INT 14-c](2.20 g, 5.31 mmol, 67.4% yield) as a white solid, m/z: [M+H]+ Calcd for C14H16BrF3NO3S, 414.0, 416.0; Found 416.2.

To a solution of (R)—N-(1-(4-bromophenyl)-2,2,2-trifluoroethyl)tetrahydro-2H-thiopyran-4-carboxamide 1,1-dioxide [INT 4-c](1 g, 2.41 mmol) in DMF (10 mL) was added Cs₂CO₃ (1.57 g, 4.82 mmol) and the reaction mixture was stirred at 25° C. for 1 h. Methyl iodide (1.02 g, 7.23 mmol) was then added at 0° C. and the reaction was stirred at 25° C. for 2 h. The reaction was quenched by adding water (50 mL), then it was extracted with EtOAc (50 mL×3). The combined organic layers were washed with brine (50 mL×2), dried over anhydrous Na₂SO₄ and concentrated under reduced pressure to give the crude product, which was purified by flash chromatography on silica gel (PE/EtOAc=1/0 to 1/1) to give (R)—N-(1-(4-bromophenyl)-2,2,2-trifluoroethyl)-N-methyltetrahydro-2H-thiopyran-4-carboxamide 1,1-dioxide [INT 14.1](700 mg, 1.63 mmol, 67.9% yield) as a colorless oil. m/z: [M+H]+ Calcd for C15H18BrF3NO3S, 428.0, 430.0; Found 430.1.

(R)-2-methylpropane-2-sulfmamide (12.1 g, 100 mmol) and 4-bromo-2-methylbenzaldehyde [INT 15-a](10 g, 50.2 mmol) were dissolved in tetrahydrofuran (50 mL) and titanium ethoxide (34.2 g, 150 mmol) was added. The mixture was stirred for 10 h at 25° C. Then the reaction mixture was poured into water (500 mL) and extracted with EtOAc (3×300 mL). The organic extracts were combined, dried under Na₂SO₄ and evaporated in vacuo. The residue was purified by flash chromatography (Hexane/MTBE=1/0 to 0/1) to give (R,E)-N-(4-bromo-2-methylbenzylidene)-2-methylpropane-2-sulfinamide [INT 15-b](10.4 g, 34.5 mmol, 68.8% yield) as a yellow solid. II NMR (400 MHz, CDCh) δ 8.76 is. 1H), 7.75 (d, J=8.7 Hz, 1H), 7.46-7.37 (m, 2H), 2.56 (s, 3H), 1.24 (s, 9H).

(R)-2-methylpropane-2-sulfinamide (18.1 g, 150 mmol) and 4-bromo-3-methylbenzaldehyde [INT 15-e](15 g, 75.3 mmol) were dissolved in tetrahydrofuran (100 mL) and titanium ethoxide (51.3 g, 225 mmol) was added. The mixture was stirred for 10 h at 60° C. Then the reaction mixture was poured into water (500 mL) and extracted with MTBE (3×300 mL). The organic extracts were re-extracted with water (3×200 mL), dried under Na₂SO₄ and evaporated in vacuo to give (R,E)-N-(4-bromo-3-methylbenzylidene)-2-methylpropane-2-sulfmamide [INT 15-f](15.2 g, 50.2 mmol, 66.9% yield) as a yellow solid. ¹H NMR (400 MHz, CDCl₃) δ 8.49 (s, 1H). 7.67 (d, J=2.2 Hz, 1H), 7.65-7.52 (m, 1H), 7.51-7.43 (m, III). 2.43 (s, 3H), 1.23 (s, 9H).

(R,E)-N-(4-bromo-3-methylbenzylidene)-2-methylpropane-2-sulfmamide [INT 15-f](21 g, 69.4 mmol) and tetrabutyiammonium triphenyldifluorosilicate (56.1 g, 104 mmol) were dissolved in THE (500 ml). Trifluoromethyltrimethylsilane (49.3 g, 347 mmol) was added dropwise at −80° C. The mixture was stirred tor 30 min at −30° C. after which an aqueous solution of NH₄Cl (300 mL) was added. The mixture was extracted with EtOAc (2×300 ml). The organic phase was dried with sodium sulfate and evaporated in vacuo at 45° C. The residue was purified by flash chromatography to obtain (R)—N—((S)-1-(4-bromo-3-methylphenyl)-2,2,2-trifluoroethyl)-2-methyipropane-2-sulfmamide [INT 15-g](18.5 g, 49.7 mmol, 71.7% yield) as a white solid, m/z: [M+H]+ Calcd for C13H18BrF3NOS, 372.0; Found 372.0. ¹H NMR (500 MHz, CDCl₃) δ 7.56 (d, J=8.2 Hz, 1H), 7.30-7.26 (m, 1H). 7.14-7.08 (m, 1H), 4.75 (p, J=7.1 Hz, 1H), 3.58 (d, J=6.4 Hz, 1H), 2.41 (s, 3H), 1.25 (s, 9H).

(R)—N—((S)-1-(4-bromo-3-methylphenyl)-2,2,2-trifluoroethyl)-2-methylpropane-2-sulfinamide [INT 15-g](10 g, 26.8 mmol) was dissolved in THE (200 mL). Lithium(1+) bis(trimethylsilyl)azanide (74.3 mL, 80.3 mmol) was added at 0° C. The mixture was stirred for 20 min at 0° C. Methyl iodide (22.7 g, 160 mmol) was added. The mixture was stirred for 10 hr at 20° C. after which an aqueous solution of NH₄Cl (200 mL) was added. The mixture was extracted with EtOAc (2×200 mL). The organic phase was dried with sodium sulfate and evaporated in vacuo at 45° C. to obtain crude (R)—N—((S)-1-(4-bromo-3-methylphenyl)-2,2,2-trifluoroethyl)-N,2-dimethylpropane-2-sulfmamide [INT 15-h](9.09 g, 23.5 mmol, 88.2% yield) as a brown oil. m/z: [M+H]+ Calcd for C14H2OBrF3NOS, 386.0; Found 386.0. ¹H NMR (400 MHz, CDCl3) δ 7.57 (d, J=8.3 Hz, 1H). 7.34 (s, 1H), 7.17 (d, J=8.6 Hz, 1H), 5.03 (q, J=8.5 Hz, 1H), 2.50-2.41 (m, 6H), 1.27 (s, 9H).

(R)—N—((S)-1-(4-bromo-3-methylphenyl)-2,2,2-trifluoroethyl)-N,2-dimethylpropane-2-sulfinamide [INT 15-h](10.7 g, 27.7 mmol) was dissolved in methanol (20 mL), after which hydrogen chloride (4 M in 1,4-dioxane, 100 mL, 2.54 mol) was added. The mixture was stirred for 10 hr at 20° C., after which it was evaporated in vacuo at 50° C. MTBE (100 mL) was added. The solid formed was filtered and washed with MTBE (50 mL) to obtain (S)-1-(4-bromo-3-methylphenyl)-2,2,2-trifluoro-N-methylethan-1-amine hydrochloride [INT 15.2](5.41 g, 16.9 mmol, 61.3% yield) as a beige solid, m/z: [M+H]+ Calcd for C10H12BrF3N 282.0, 284.0; Found 284.0. ¹H NMR (500 MHz, DMSO-d6) δ 10.51 (s, 2H), 7.76 (d, J=8.3 Hz, 1H), 7.63 (d, J=2.2 Hz, 1H), 7.41 (dd, J=8.3, 2.2 Hz, 1H), 5.42 (s, 1H), 2.43 (s, 3H), 2.37 (s, 3H).

Phosphorus oxychloride (788 mg, 5.14 mmol) was added to a solution of [(1S)-1-(4-bromo-2-methylphenyl)-2;2,2-trifluoroethyl](methyl)amine hydrochloride [INT 15.1](500 mg, 1.56 mmol) and 1,1~dioxo-1λ⁶-thiane-4-carboxylic acid [INT 4-a](833 mg, 4.68 mmol) in pyridine (3 mL) at 0° C. The reaction mixture was stirred overnight. An aqueous solution of sodium bicarbonate (3 mL) was added, and the mixture was extracted with EtOAc (3×10 mL) and washed with NaHSOr (3×10 mL). The combined organic layers were dried over anhydrous NaiSCb and evaporated under reduced pressure. The crude product was purified by HPLC (see conditions below) to give N-[(1S)-1-(4-bromo-2-methylphenyl)-2,2,2-trifluoroethyl]-N-methyl-1,1-dioxo-1λ⁶-thiane-4-carboxamide [INT 16.3](132 mg, 0.2985 mmol, 19.1% yield) as a pink solid, m/z; [M+H]+ Calcd for C16H2OBrF3NO3S, 442.0, 444.0; Found 444.0.

[(1S)-1-(4-bromo-3-methylphenyl)-2,2,2-trifluoroethyl](methyl)amine hydrochloride [INT (0.5 g, 1.56 mmol) and 1,1-dioxo-1λ⁶-thiane-4-carboxylic acid [INT 4-a](833 mg, 4.68 mmol) were mixed in pyridine (2 mL). Phosphorus oxychloride (788 mg, 5.14 mmol) was added. The mixture was stirred for 10 hr at 90° C. EtOAc (20 mL) was added, and the mixture was washed with an aqueous NaHSOr solution (3×5 mL). The organic phase was dried with sodium sulfate and evaporated in vacuo at 45° C. to obtain crude N-[(1S)-1-(4-bromo-3-methylphenyl)-2,2,2-trifluoroethyl]-N-methyl-1,1-dioxo-1λ⁶-thiane-4-carboxamide [INT 17.3](656 mg, 1.48 mmol) as a yellow solid, m/z: [M+H]+ Calcd for C16H2OBrF3NO3S, 442.0; Found 442.0.

To a solution of N-[(1R)-1-(4-bromophenyl)-2,2,2-trifluoroethyl]-N-methyl-1,1-dioxo-1λ⁶-thiane-4-carboxamide [INT 14.1](50 mg, 116 mol) and 2-chloro-7-[(1 S)H-methoxyethyl]-[1,2,4]triazolo[1,5-a]pyrimidin-6-amine hydrochloride [INT 1.1](31.6 mg, 139 mol) in dioxane (2 mL) was added Pd₂(dba)₃ (10.6 mg, 11.6 μiηol), Cs₂CO₃ (113 mg, 348 mol), and xantphos (13.4 mg, 23.2 μτηol). The reaction mixture was stirred at 100° C. for 2 h under N2. The reaction mixture was concentrated under reduced pressure to give the crude product, which was purified by prep-HPLC (column: Boston Green ODS 150*30 mm*5 pm, table: 24-64% B (A=water (0.05% ammonia hydroxide)), B=acetonitrile), flow rate: 30 mL/min, UV Detector 220 nm) to afford N-[(1R)-1-[4-({2-chloro-7-[(1S)-1-methoxyethyl]-[L2,4]triazolo[1,5-a]pyrimidin-6-yl}amino)phenyl]-2,2,2-trifluoroethyl]-N-methyl-1,1-dioxo-1λ⁶-thiane-4-carboxamide [Compound 1.26](16.4 mg, 28.5 μmol, 24.6% yield) as a yellow dry powder, m/z: [M+H]+ Calcd for C23H27ClF3N604S 575.1; Found 575.3. ¹H NMR (400MHZ, DMSO-d6) δ=8.83 (s, 1H), 8.07-7.97 (m, 1H), 7.29-7.17 (m, 2H), 7.03-6.92 (m, 2H), 6.49-6.05 (m, 1H), 5.16 (q, J=6.8 Hz, 1H), 3.26-3.19 (m, 2H), 3.16 (s, 3H), 3.13-3.08 (m, 2H), 2.90 (s, 3H), 2.65 (s, 1H), 2.10-1.95 (m, 4H), 1.59 (d, J=6.8 Hz, 3H).

A mixture of 2-chloro-7-[(1R)-1-methoxyethyl]-[1,2,4]triazolo[1,5-a]pyrimidin-6-amine hydrochloride|INT 1.3] (60 mg, 227 μηiol), N-[(1R)-1-(4-bromophenyl)-2,2,2-trifluoroethyl]-N-methyl-1,1-dioxo-1λ⁶-thiane-4-carboxamide [1MT 14.1](97.2 mg, 227 μηiol), Pd₂(dba)₃ (20.7 mg, 22.7 mol), Xantphos (26.2 mg, 45.4 mol), and Cs₂CO₃ (221 mg, 681 mol) in dioxane (2 ml) was stirred at 100° C. for 3 hr under N₂ atmosphere. The mixture was concentrated under reduced pressure to afford the crude product, which was purified by flash chromatography on silica gel (methanol/dichloromethane=0/1 to 1/20). The resulting product was purified by prep-HPLC (column: YMC Triart C18 250*50 mm*7 um, table: 26-66% B (A=water (0.05% ammonia hydroxide v/v)), B=acetonitrile), flow rate: 60 mL/min, UV Detector 220 nm) and prep-TLC (SiO2, dichloromethane:mefhano{=20:1) to afford N—[(IR)—I-[4-({2-chloro-7-[(1R)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide [Compound 1.27](5.20 mg, 9.04 mol, 4.0% yield) as a white dry powder, m/z: [M+H.]+ Calcd for C23H27ClF3N6O4S, 575.1; Found 575.3. ¹H NMR (400 MHz, CD₃OD) δ=8.86 (s, 1H), 7.30 (d, J=8.4 Hz, 2H), 7.06 (d, J=8.4 Hz, 2H), 6.52 (q, J=9.2 Hz, 1H), 5.36 (q, J=6.8 Hz, 1H), 3.36 (s, 3H), 3.25-3.10 (m, 5H), 3.02-2.75 (m, 3H), 2.35-2.12 (m, 4H), 1.64 (d, J=6.8 Hz, 3H).

A mixture of N-[(1S)-1-(4-bromo-2-methylphenyl)-2,2,2-trifluoroethyl]-N-methyl-1,1-dioxo-1λ⁶-thiane-4-carboxamide [INT 16.3](100 mg, 0.2260 mmol), 2-chloro-7-[(1S)-1-methoxyethyl]-[1,2,4]triazolo[1,5-a]pyrimidin-6-amine [free base of INT 1.I](51.4 mg, 226 mol), Cs₂CO₃ (220 mg, 678 μmol), and dioxane (3 mL) was purged with argon. Then xantphos (26.1 mg, 45.2 μmol) and Pd₂(dba)₃ (20.6 mg, 22.6 μmol) were added and the reaction mixture was stirred at 100° C. for 10 h. After cooling, the reaction mixture was diluted with EtOAc (30 mL) and concentrated under reduced pressure. The resulting residue was purified by HPLC to obtain N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methyl-1,1-dioxo-1λ⁶-thiane-4-carboxamide [Compound 1.30](12.5 mg, 0,02128 mmol, 9.4% yield) as a yellow solid, m/z: [M+H]+ Calcd for C24H29ClF3N6O4S, 589.2; Found 589.0. ¹H NMR (500 MHz, DMSO-d6) δ=8,80 (s, 1H), 7.90 (s, 1H), 7.29 (d, J=8.3 Hz, 1H). 6.87-6.75 (m, 2H), 6.43 (q, J=9.1 Hz, 1H), 5.13 (q, J=6.8 Hz, 1H), 3.27-3.14 (m, 2H), 3.15 (s, 3H), 3.12-3.04 (m, 3H), 2.79 (s, 3H), 2.15-2.03 (m, 2H). 2.01 (s, 3H), 1.99-1.94 (m, 2H), 1.57 (d, J=6.7 Hz, 3H).

N-[(1S)-1-(4-bromo-3-methylphenyl)-2,2,2-trifluoroethyl]-N-methyl-1,1-dioxo-1λ⁶-thiane-4-carboxamide [INT 17.3](0.1 g, 0.2260 mmol), 2-chloro-7-[(1S)-1-methoxyethyl]-[1,2,4]triazolo[1,5-a]pyrimidin-6-amine [free base of INT 1.1](51.4 mg, 226 μmol), Cs₂CO₃ (220 mg, 678 umol), and xantphos (13.0 mg, 22.6 mol) were mixed in dioxane (2 mL) and the reaction mixture was degassed with argon for 5 min. Pd₂(dba)₃ (10.3 mg, 11.3 mol) was added. Then the reaction mixture was degassed with argon for 5 min and stirred at 100° C. for 10 h. The reaction mixture was cooled to rt and the solid was filtered off. The filtrate was purified by HPLC (see conditions below) to obtain N-[(1S)-1-[4-({2-chloro-7-[(1S)-1-methoxyethyl]-[1,2,4]tri azolo [1,5-a]pyrimidin-6-yl}amino)-3-methyl phenyl]-2,2,2-trifluoroethyl]-N-methyl 1,1-dioxo-1λ⁶-thiane-4-carboxamide [Compound 1.33](20.7 mg, 0.03515 mmol, 15.5% yield) as a yellow solid, m/z: [M+H]+ Calcd for C24H29ClF3N6)4S, 589.2; Found 589.0. ¹H NMR (600 MHz, DMSO-d6) δ=8.74 (s, 1H), 7.2.4 (s, 1H), 7.15 (s, 1H), 7.03 (d, J=8.6 Hz, 1H), 6.84 (d, J=8.4 Hz, 1H), 6.41 (q, J=9.4 Hz, 1H), 5.16 (q, J=6.7 Hz, 1H), 3.21 (d, J=13.9 Hz, 3H), 3.18-3.05 (m, 5H), 2.89 (s, 3H), 2.34-2.28 (m, 3H), 2.11-1.95 (m, 4H), 1.54 (d, J=6.7 Hz, 3H).

### Synthesis of 2-chloro-7-[(1S)-1-methoxyethyl]-[1,2,4]triazolo[1,5-a]pyrimidin-6-amine hydrochloride (Intermediate 1.1)

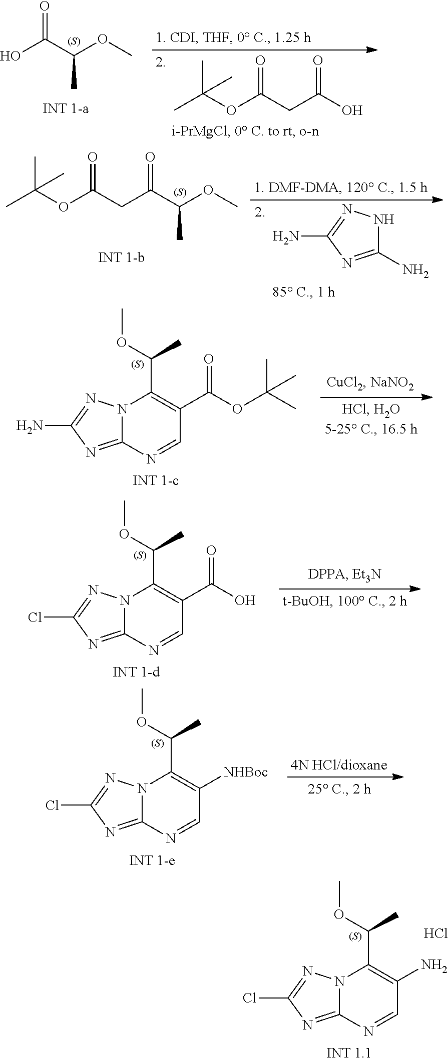

### Synthesis of tert-butyl (S)-4-methoxy-3-oxopentanoate (INT 1-b)

A solution of (S)-2-methoxypropanoic acid [INT 1-a](20 g, 192 mmol) in anhydrous tetrahydrofuran (342 mL) was cooled to 0° C. Carbonyldiimidazole (30.6 g, 189 mmol) was added at 0° C. by several portions and the mixture was stirred at this temperature for 1.25 h. In a separate flask, to a solution of 3-(tert-butoxy)-3-oxopropanoic acid (46.1 g, 288 mmol) in anhydrous tetrahydrofuran (342 mL) was added magnesium(1+) 1-methylethyl chloride (249 mL, 499 mmol, 2M in THF) at 0° C. and the mixture was stirred at room temperature for 1.25 h. Then, this solution was added to the acyl imidazole solution via a cannula at 0° C. and the resulting mixture was stirred at room temperature overnight. The reaction mixture was cooled to 0° C. and quenched by adding 10% aqueous citric acid, extracted with EtOAc, washed with saturated aqueous NaHCO₃, dried over anhydrous Na₂SO₄ and concentrated under reduced pressure to give the crude product which was purified by flash chromatography on silica gel (Acetone/hexane=0/1 to 1/9) to give tert-butyl (S)-4-methoxy-3-oxopentanoate [INT 1-b](30.0 g, 148 mmol, 55.6%) as an oil.

### Synthesis of tert-butyl (S)-2-amino-7-(1-methoxyethyl)-[1,2,4]triazolo[1,5-a]pyrimidine-6-carboxylate (INT 1-c)

A solution of tert-butyl (S)-4-methoxy-3-oxopentanoate [INT 1-b](25 g, 123 mmol) and (dimethoxymethyl)dimethylamine (11.1 mL, 83.6 mmol) was heated at 120° C. for 1.5 h. The mixture was cooled to room temperature and 4H-1,2,4-triazole-3,5-diamine (12.1 g, 123 mmol) followed by ethanol (123 mL) were added, and the mixture was heated at 85° C. for 1 h. After completion, the mixture was concentrated under reduced pressure and recrystallized from EtOH/water (1:1, 600 mL), filtered, and the filter cake was washed with 30% EtOH/water, followed by MTBE to give tert-butyl 2-amino-7-(1-methoxyethyl)-[1,2,4]triazolo[1,5-a]pyrimidine-6-carboxylate (12.7 g, 43.2 mmol, 52%) as a solid. The filtrate was concentrated under reduced pressure to remove MTBE and the solid was filtered and washed with hexane to get more give tert-butyl (S)-2-amino-7-(1-methoxyethyl)-[1,2,4]triazolo[1,5-a]pyrimidine-6-carboxylate [INT 1-c](5.3 g, 18.0 mmol, 23%) as an solid. The total 18.0 g, 75% yield. The chiral HPLC showed 96.9% ee. ¹H NMR (400 MHz, CDCl₃) δ=8.75 (s, 1H), 5.40 (q, J=6.8 Hz, 1H), 4.95 (br s, 2H), 3.30 (s, 3H), 1.75 (d, J=6.8 Hz, 3H), 1.62 (s, 9H).

### Synthesis of 2-chloro-7-[(1S)-1-methoxyethyl]-[1,2,4]triazolo[1,5-a]pyrimidine-6-carboxylic acid INT 1-d)

To a mixture of tert-butyl 2-amino-7-[(1S)-1-methoxyethyl]-[1,2,4]triazolo[1,5-a]pyrimidine-6-carboxylate [INT 1-c](1.2 g, 4.09 mmol) and Copper(II) chloride dihydrate (173 mg, 1.02 mmol) in conc. HCl (20 mL) was added a solution of sodium nitrite (338 mg, 4.90 mmol) in H₂O (5 mL) at 5° C. with ice bath and the mixture was stirred at 5° C. for 30 min. Then the mixture was warmed to 25° C. and stirred for 16 hr. Water (100 mL) was added and 1 N NaOH aqueous solution was added to adjust the pH to 3-4. The mixture was extracted with CHCl₃:i-PrOH=3:1 (100 mL×3) and the combined organic layers were dried over anhydrous Na₂SO₄ and concentrated under reduced pressure to give 2-chloro-7-[(1S)-1-methoxyethyl]-[1,2,4]triazolo[1,5-a]pyrimidine-6-carboxylic acid [INT 1-d](962 mg, 92.4% yield) as a solid. m/z: [M+H]+ Calcd for C9H10C1N4O3 257.0; Found 256.9. ¹H NMR (400 MHz, DMSO-d₆) δ=14.00 (br s, 1H), 9.07 (s, 1H), 5.39 (q, J=6.4 Hz, 1H), 3.21 (s, 3H), 1.63 (d, J=6.4 Hz, 3H).

### Synthesis of tert-butyl N-{2-chloro-7-[(1S)-1-methoxyethyl]-[1,2,4]triazolo[1,5-a]pyrimidin-6-yl}carbamate (INT 1-e)

To a solution of 2-chloro-7-[(1S)-1-methoxyethyl]-[1,2,4]triazolo[1,5-a]pyrimidine-6-carboxylic acid [INT 1-d](1.3 g, 5.06 mmol) in t-BuOH (10 mL) was added {[azido(phenoxy)phosphoryl]oxy}benzene (2.08 g, 7.58 mmol) and triethylamine (1.02 g, 10.1 mmol) and the mixture was stirred at 100° C. for 2 h under N₂ atmosphere. The mixture was concentrated under reduced pressure to afford the crude product which was purified by flash chromatography on silica gel (EtOAc/Petroleum ether=1/10 to 1/5) to afford tert-butyl N-{2-chloro-7-[(1S)-1-methoxyethyl]-[1,2,4]triazolo[1,5-a]pyrimidin-6-yl}carbamate [INT 1-e](420 mg, 25.4% yield) as a solid. m/z: [M+H]+ Calcd for C13H19ClN5O3 328.1; Found 328.0. ¹H NMR (400 MHz, CDCl3) δ=9.62 (br s, 1H), 8.05 (s, 1H), 5.45 (q, J=6.8 Hz, 1H), 3.48 (s, 3H), 1.63 (d, J=6.8 Hz, 3H), 1.56 (s, 9H).

### Synthesis of 2-chloro-7-[(1S)-1-methoxyethyl]-[1,2,4]triazolo[1,5-a]pyrimidin-6-amine hydrochloride (INT 1.1)

A mixture of tert-butyl N-{2-chloro-7-[(1S)-1-methoxyethyl]-[1,2,4]triazolo[1,5-a]pyrimidin-6-yl}carbamate [INT 1-e](420 mg, 1.28 mmol) in 4N HCl/dioxane (5 mL) was stirred at 25° C. for 2 h. LCMS showed the reaction was completed and one new peak with desired MS was detected (Rt=0.611 min, m/z: 227.8 [M+H]+). The mixture was concentrated under reduced pressure to afford 2-chloro-7-[(1S)-1-methoxyethyl]-[1,2,4]triazolo[1,5-a]pyrimidin-6-amine hydrochloride [INT 1.1](400 mg, crude) as a solid.

### Synthesis of tetrahydro-2H-thiopyran-4-carbonyl chloride 1,1-dioxide (Intermediate 4.1)

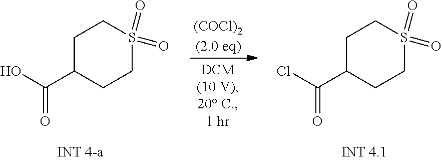 [SMILES: O=C(O)C1CCS(=O)(=O)CC1 ; "(COCl)2(2.0 eq)" ; O=C(Cl)C1CCS(=O)(=O)CC1]

To a solution of tetrahydro-2H-thiopyran-4-carboxylic acid 1,1-dioxide [INT 4-a](41.0 g, 230 mmol, 1.0 eq) in DCM (410 mL) was added (COCl)₂ (58.4 g, 460 mmol, 40.3 mL, 2.0 eq) and DMF (168 mg, 2.30 mmol, 177 μL, 0.01 eq) at 0° C. under N₂. The mixture was warmed to 20° C. and stirred at 20° C. for 2 h. The suspension turned to clear, which showed the most of starting material was consumed. The reaction mixture was concentrated in vacuum to give the crude product as a solid, which was concentrated by oil pump to remove the solvent residue to give tetrahydro-2H-thiopyran-4-carbonyl chloride 1,1-dioxide [INT 4.1](46.5 g, crude) as a solid.

### Synthesis of (S)—N-(1-(4-bromophenyl)-2,2,2-trifluoroethyl)-N-methyltetrahydro-2H-thiopyran-4-carboxamide 1,1-dioxide (Intermediate 5.1)

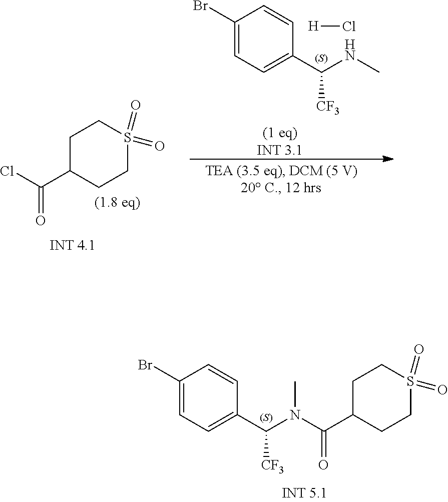

To a solution of (S)-1-(4-bromophenyl)-2,2,2-trifluoro-N-methylethan-1-amine hydrochloride [INT 3.1](39.0 g, 128 mmol, 1.0 eq, HCl) and TEA (45.7 g, 451 mmol, 62.8 mL, 3.5 eq) in DCM (200 mL) was added tetrahydro-2H-thiopyran-4-carbonyl chloride 1,1-dioxide [INT (45.3 g, 231 mmol, 1.8 eq) at 0-10° C. The mixture was stirred at 20° C. for 12 h. The mixture was separated to give the organic layer and the aqueous layer was extracted with DCM (100 mL). The combined organic layer was concentrated in vacuum to give the crude product as an oil. The crude product was purified by column chromatography on silica gel with petroleum ether/ethyl acetate (15/1-3/1) to give (S)—N-(1-(4-bromophenyl)-2,2,2-trifluoroethyl)-N-methyltetrahydro-2H-thiopyran-4-carboxamide 1,1-dioxide [INT 5.1](26.0 g, 60.7 mmol, 47.4% yield, 100% purity) as a solid. ¹H NMR (400 MHz, CDCl3) δ 2.25-2.37 (m, 1H), 2.38-2.40 (m, 3H), 2.88-3.00 (m, 6H), 3.30-3.31 (m, 1H), 3.22-3.45 (m, 1H), 6.56-6.63 (m, 1H), 7.23 (d, J=8.00 Hz, 2H), 7.55 (d, J=8.40 Hz, 2H).

SFC: Rt=1.21 min, 100.0% ee; Column: Chiralpak AD-3, 50×4.6 mm I.D., 3 um; Mobile phase: A: CO₂, B: MeOH (0.05% IPAm, v/v); Flow rate: 3.4 mL/min; Column temp.: 35° C.

LCMS: Rt=2.431 min, 100% purity, m/z=428.0, 430.0 (M+1)+. The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (Sum particles). Detection methods are diode array (DAD) as well as positive electrospray ionization. MS range was 100-1000.

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide (also known as N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide) (Compound 1.1)

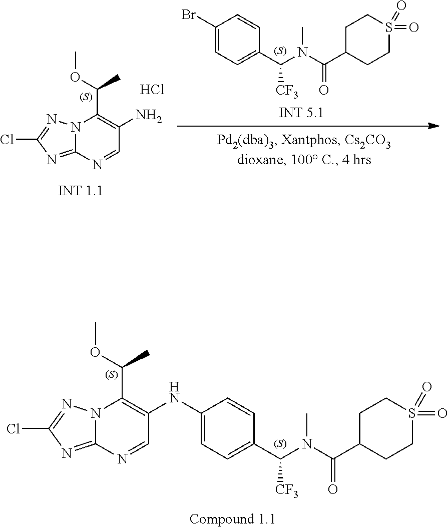

A mixture of 2-chloro-7-[(1S)-1-methoxyethyl]-[1,2,4]triazolo[1,5-a]pyrimidin-6-amine hydrochloride [INT 1.1](460 mg, 1.74 mmol), N-[(1S)-1-(4-bromophenyl)-2,2,2-trifluoroethyl]-N-methyl-1,1-dioxo-1)⁶-thiane-4-carboxamide [INT 5.1](779 mg, 1.82 mmol), Pd₂(dba)₃ (159 mg, 174 μmol), Xantphos (201 mg, 348 μmol) and Cs₂CO₃ (1.70 g, 5.22 mmol) in dioxane (6 mL) was stirred at 100° C. for 4 h. The mixture was concentrated under reduced pressure to afford the crude product which was purified by flash chromatography on silica gel (Methanol/Dichloromethane=0/1 to 1/10) and Prep-HPLC (column: YMC Triart C18 250*50 mm*7 μm, table: 29-58% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide [Compound (211 mg, 368 μmol) as a dry powder. m/z: [M+H]+ Calcd for C23H27ClF3N6O4S, 575.2; Found 575.3. ¹H NMR (400 MHz, DMSO-d₆) δ=8.80 (s, 1H), 8.02-7.94 (m, 1H), 7.26-7.12 (m, 2H), 6.99-6.89 (m, 2H), 6.45-6.02 (m, 1H), 5.12 (q, J=6.8 Hz, 1H), 3.25-3.14 (m, 3H), 3.12 (s, 3H), 3.10-3.05 (m, 2H), 2.86 (s, 3H), 2.08-1.94 (m, 4H), 1.55 (d, J=6.8 Hz, 3H). Compound 1.1 was determined to have a chiral purity of at least 89%.

Synthesis of other MALT1 inhibitors is described in WO 2022/081967 (PCT/US21/55173) which is incorporated by reference in its entirety for all purposes.

### Synthesis of MALT1 Inhibitors of Formula Ia

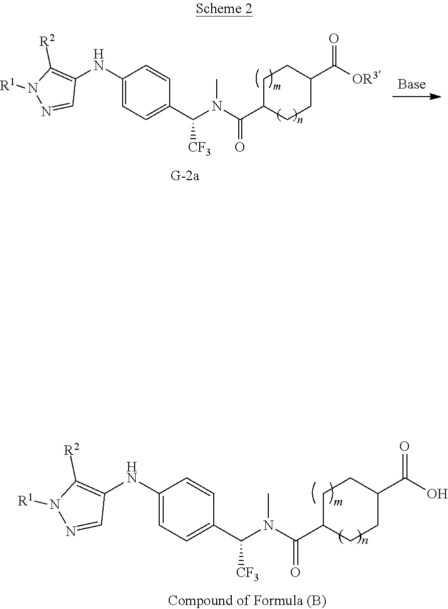

Starting material G-2a is treated with base (LiOH·H2O or NaOH) to provide a compound of Formula (B). R2 is phenyl, pyridyl, or pyridazine; R1 is CF3 or (S)-methoxyethane; and m and n are each independently 0 or 1. R3′ is methyl or benzyl.

### Synthesis of 1-(pyridin-2-yl)-5-(trifluoromethyl)-1H-pyrazol-4-amine

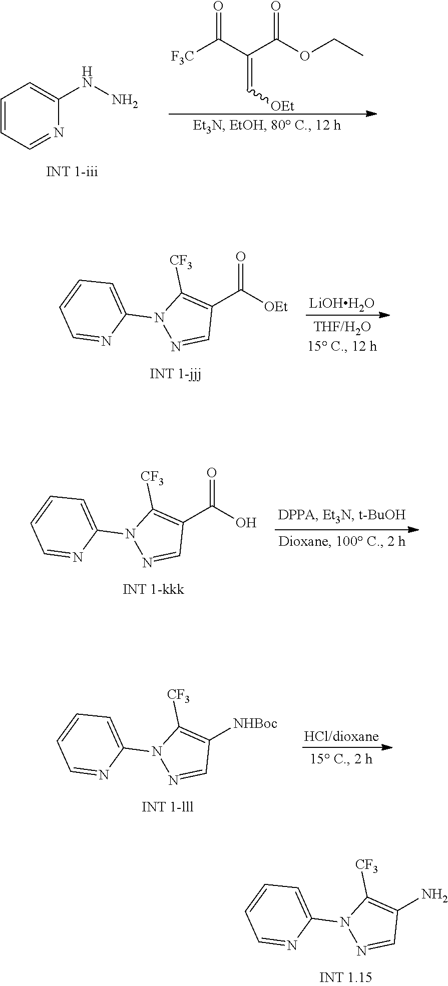

To a solution of ethyl 2-(ethoxymethylidene)-4,4,4-trifluoro-3-oxobutanoate (5 g, 20.8 mmol) and 2-hydrazinylpyridine [INT 1-iii](2.26 g, 20.8 mmol) in EtOH (30 mL) was added triethylamine (2.10 g, 20.8 mmol). The mixture was stirred at 80° C. for 12 hr. The reaction mixture was concentrated under reduced pressure to give the crude product, which was purified by flash chromatography on silica gel (EtOAc/PE=0/1 to 10/1) to give ethyl 1-(pyridin-2-yl)-5-(trifluoromethyl)-1H-pyrazole-4-carboxylate [INT 1-jjj](5.00 g, 17.5 mmol, 84.3% yield) as yellow oil. m/z: [M+H]+ Calcd for C12H11F3N3O2 286.1; Found 285.9. ¹H NMR (400 MHz, DMSO-d6) δ=8.64-8.59 (m, 1H), 8.34 (s, 1H), 8.19-8.12 (m, 1H), 7.80 (dd, J=0.8, 8.0 Hz, 1H), 7.68-7.63 (m, 1H), 4.33 (q, J=7.2 Hz, 2H), 1.31 (t, J=7.2 Hz, 3H).

To a mixture of ethyl 1-(pyridin-2-yl)-5-(trifluoromethyl)-1H-pyrazole-4-carboxylate [INT 1-jjj](1 g, 3.50 mmol) in THE (6 mL) and H₂O (2 mL) was added lithium hydroxide monohydrate (440 mg, 10.5 mmol). The reaction mixture was stirred at 15° C. for 12 hours. The reaction mixture was concentrated under reduced pressure. The residue was acidified with 1 M HCl to pH=4. The reaction mixture was quenched by the addition of H₂O (20 mL) and extracted with EtOAc (20 mL×2). The combined organic layers were washed with brine (20 mL), dried over Na₂SO₄, filtered and concentrated under reduced pressure to give 1-(pyridin-2-yl)-5-(trifluoromethyl)-1H-pyrazole-4-carboxylic acid [INT 1-kkk](800 mg, 3.11 mmol, 88.8% yield) as a yellow solid. ¹H NMR (400 MHz, DMSO-d6) δ=13.43 (br s, 1H), 8.60 (td, J=0.8, 4.8 Hz, 1H), 8.28 (s, 1H), 8.14 (dt, J=2.0, 7.6 Hz, 1H), 7.79 (d, J=8.0 Hz, 1H), 7.64 (ddd, J=0.8, 4.8, 7.6 Hz, 1H).

A mixture of 1-(pyridin-2-yl)-5-(trifluoromethyl)-1H-pyrazole-4-carboxylic acid [INT 1-kkk](400 mg, 1.55 mmol), triethylamine (784 mg, 7.75 mmol), and diphenylphosphoryl azide (564 mg, 2.32 mmol) in t-BuOH (3 mL) and dioxane (3 mL) was stirred at 100° C. for 2 hr. The reaction mixture was concentrated under reduced pressure to give the crude product, which was purified by flash chromatography on silica gel (Petroleum ether/EtOAc=1/0 to 1/1) to give tert-butyl N-[1-(pyridin-2-yl)-5-(trifluoromethyl)-1H-pyrazol-4-yl]carbamate [INT 1-111](470 mg, 1.43 mmol, 92.5% yield) as a white solid. m/z: [M+H]+ Calcd for C14H16F3N402 329.1; Found 328.9. ¹H NMR (400 MHz, DMSO-d6) δ=9.14 (br s, 1H), 8.56-8.51 (m, 1H), 8.06 (dt, J=1.8, 7.6 Hz, 1H), 7.95 (s, 1H), 7.77 (d, J=8.0 Hz, 1H), 7.55-7.46 (m, 1H), 1.46 (s, 9H).

To a solution of tert-butyl N-[1-(pyridin-2-yl)-5-(trifluoromethyl)-1H-pyrazol-4-yl]carbamate [INT 1-111](470 mg, 1.43 mmol) in 4 M HCl/dioxane (4 mL) was stirred at 15° C. for 2 h. The reaction mixture was concentrated under reduced pressure to give 1-(pyridin-2-yl)-5-(trifluoromethyl)-1H-pyrazol-4-amine [INT 1.15](260 mg, 1.13 mmol, 79.7% yield) as a white solid. m/z: [M+H]+ Calcd for C9H8F3N4 229.1; Found 228.9. ¹H NMR (400 MHz, DMSO-d6) δ=8.48 (dd, J=1.2, 4.8 Hz, 1H), 8.01 (dt, J=1.6, 7.6 Hz, 1H), 7.76-7.72 (m, 2H), 7.65 (br s, 2H), 7.43 (dd, J=4.8, 6.8 Hz, 1H).

### Synthesis of (R)—N—((S)-1-(4-bromophenyl)-2,2,2-trifluoroethyl)-2-methylpropane-2-sulfinamide

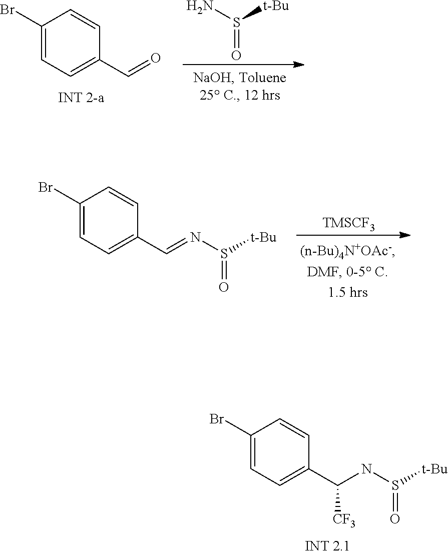

To a solution of 4-bromobenzaldehyde [INT 2-a](100 g, 541 mmol, 1.0 eq) in toluene (500 mL) was added (R)-2-methylpropane-2-sulfinamide (72.1 g, 595 mmol, 1.1 eq) at 25° C. The mixture was stirred at 25° C. for 15 mins. Then to above reaction was added NaOH (21.6 g, 541 mmol, 1.0 eq) and the mixture was stirred at 25° C. for 12 hours. Na₂SO₄ (50 g) was added to the mixture and stirred for 20 mins. Four reaction mixtures were combined and filtered through celite to give the filtrate, which was concentrated in vacuum to give the crude product as an oil. The crude product was dissolved in petroleum ether (1.0 L) and stirred at −50° C. for 1.0 hour, filtered to give (R,E)-N-(4-bromobenzylidene)-2-methylpropane-2-sulfinamide [INT 2-b](620 g, 2.15 mol, 99.5% yield) as a solid.

To a solution of (R,E)-N-(4-bromobenzylidene)-2-methylpropane-2-sulfinamide [INT 2-b](206 g, 715 mmol, 1.0 eq) and tetrabutylammonium acetate (216 g, 715 mmol, 218 mL, 1.0 eq) in DMF (1.4 L) was added TMSCF₃ (259 g, 1.82 mol, 2.5 eq) at 0° C. The mixture was stirred at 5° C. for 1.5 hours. This process was repeated 2 times and the three reaction mixtures were combined for work-up. The mixture was poured into saturated NH₄Cl solution (13.0 L) and stirred for 10 mins to give a suspension. The suspension was filtered and the filter cake was washed with water (5.0 L). The filter cake was triturated with MTBE/Petroleum ether (v/v=1:4, 2.0 L) and to give the product as a solid and the mother liquid was concentrated in vacuum to give the crude product as an oil which was purified by column chromatography on silica gel with petroleum ether/ethyl acetate (10/1-1/1) to give (R)—N—((S)-1-(4-bromophenyl)-2,2,2-trifluoroethyl)-2-methylpropane-2-sulfinamide [INT 2.1](389 g, 1.09 mol, 50.6% yield) as a solid. ¹H NMR (400 MHz, CDCl₃) δ=1.25 (s, 9H), 3.64 (d, J=6.40 Hz, 1H), 4.79-4.83 (m, 1H), 7.32 (d, J=8.40 Hz, 2H), 7.56 (d, J=6.40 Hz, 2H).

### Synthesis of (S)-1-(4-bromophenyl)-2,2,2-trifluoro-N-methylethan-1-amine

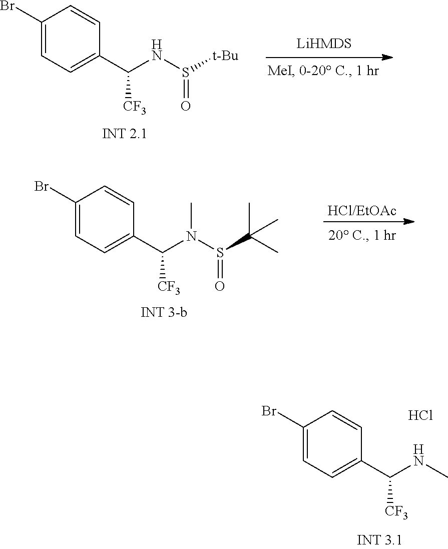

To a solution of LiHMDS (1.0 M, 838 mL, 3.0 eq) was added (R)—N—((S)-1-(4-bromophenyl)-2,2,2-trifluoroethyl)-2-methylpropane-2-sulfinamide [INT 2.1](100 g, 279 mmol, 1.0 eq) at 0-10° C. and the resulting mixture was stirred at 0-10° C. for 0.5 hour. To the above mixture was added Mel (119 g, 838 mmol, 52.1 mL, 3.0 eq) at 0-10° C. and the reaction was stirred at 25° C. for 1 hour. The process was repeated 2 times and the three combined reaction mixtures were poured into saturated aqueous NH₄Cl (3.0 L) and diluted with EtOAc (1.0 L). The mixture was separated to give the organic layer and the aqueous layer was extracted with EtOAc (500 mL). The combined organic layer was washed with saturated NaCl (1.0 L) and dried with Na₂SO₄, filtered and concentrated in vacuum to give the crude product as an oil. The crude product was purified by column chromatography on silica gel with petroleum ether/ethyl acetate (15/1-1/1) to give (R)—N—((S)-1-(4-bromophenyl)-2,2,2-trifluoroethyl)-N,2-dimethylpropane-2-sulfinamide [INT 3-b](161 g, 432.5 mmol, 51.6% yield) as an oil.

To the mixture of (R)—N—((S)-1-(4-bromophenyl)-2,2,2-trifluoroethyl)-N,2-dimethylpropane-2-sulfinamide [INT 3-b](202 g, 543 mmol, 1.0 eq) in EtOAc (600 mL) was added HCl/EtOAc (4.0 M, 2.02 L, 14.9 eq) slowly. The above mixture was stirred at 20° C. for 1 hour. The reaction mixture was filtered to give a solid and eluted with EtOAc (200 mL) and the mother liquid was concentrated in vacuum to give a solid. The solid was purified by column chromatography on silica gel with petroleum ether/ethyl acetate (10/1-1/0) and combined with the filter cake and concentrated by oil pump at 45° C. for 1 hour to remove the solvent residue to give (S)-1-(4-bromophenyl)-2,2,2-trifluoro-N-methylethan-1-amine hydrochloride [INT 3.1](115 g, 378 mmol, 69.6% yield, 100% purity, HCl) as a solid. ¹H NMR (400 MHz, DMSO-d₆) δ=2.45 (s, 3H), 5.51 (s, 1H), 7.62 (d, J=8.4 Hz, 2H), 7.78 (d, J=8.40 Hz, 2H), 10.59 (s, 2H).

SFC: Rt=0.776 min, 99.98% ee; Column: Chiralpak AD-3, 100×4.6 mm, I.D., 3 μm; Mobile phase: A: CO₂, B: MeOH (0.05% IPAm); Gradient: A:B=97:3; Flow rate: 3 mL/min; Column temp.: 35° C.

LCMS: Rt=1.755 min, 100.0% purity, m/z=268.0, 270.0 (M+1)+. The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 μm particles). Detection methods are diode array (DAD) as well as positive electrospray ionization. MS range was 100-1000.

### Synthesis of Methyl (1r,4r)-4-(chlorocarbonyl)cyclohexane-1-carboxylate

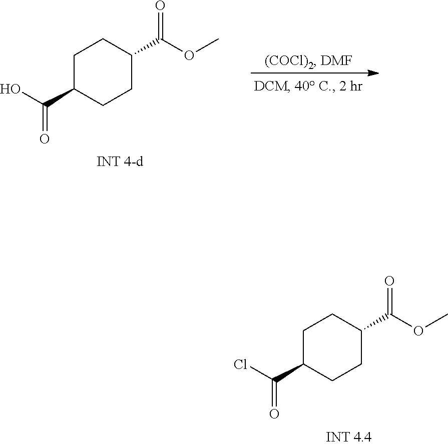

To a mixture of (1r,4r)-4-(methoxycarbonyl)cyclohexane-1-carboxylic acid [INT 4-d](1.45 g, 7.78 mmol) in dichloromethane (10 mL) was added oxalyl dichloride (2.93 g, 23.3 mmol) and DMF (56.8 mg, 778 μmol) slowly and the mixture was stirred at 40° C. for 2 hr. The mixture was concentrated under reduced pressure to afford the crude methyl (1r,4r)-4-(chlorocarbonyl)cyclohexane-1-carboxylate [INT 4.4](1.59 g, 7.76 mmol) as a yellow gum.

### Synthesis of Methyl (1S,4r)-4-(((S)-1-(4-bromophenyl)-2,2,2-trifluoroethyl)(methyl)carbamoyl)cyclohexane-1-carboxylate

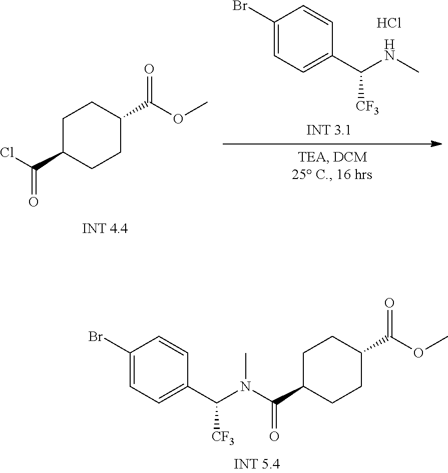 [SMILES: COC(=O)[C@H]1CC[C@H](C(=O)Cl)CC1 ; CN[C@@H](c1ccc(Br)cc1)C(F)(F)F ; Cl ; COC(=O)[C@H]1CC[C@H](C(=O)N(C)[C@@H](c2ccc(Br)cc2)C(F)(F)F)CC1]

To a mixture of methyl (1r,4r)-4-(carbonochloridoyl)cyclohexane-1-carboxylate [INT 4.4](1.59 g, 7.76 mmol) and Et₃N (2.65 g, 26.2 mmol) in dichloromethane (6 mL) was added a solution of [(1S)-1-(4-bromophenyl)-2,2,2-trifluoroethyl](methyl)amine hydrochloride [INT 3.1](1.6 g, 5.25 mmol) in dichloromethane (6 mL) and the mixture was stirred at 25° C. for 16 hr. Water (30 mL) was added and the mixture was extracted with dichloromethane (30 mL×2). The combined organic layers were washed with brine (30 mL), dried over anhydrous Na₂SO₄, filtered and concentrated under reduced pressure to give the crude product which was purified by flash chromatography on silica gel (EtOAc/Petroleum ether=1/10 to 1/5) to give methyl (1 S,4r)-4-(((S)-1-(4-bromophenyl)-2,2,2-trifluoroethyl)(methyl)carbamoyl)cyclohexane-1-carboxylate [INT 5.4](1.10 g, 2.52 mmol, 32.5% yield) as yellow oil. m/z: [M+H]+ Calcd for C18H22BrF3NO3 436.1, 438.1; Found 438.0.

### Synthesis of methyl (1r,4r)-4-{methyl[(1S)-2,2,2-trifluoro-1-(4-{[1-(pyridin-2-yl)-5-(trifluoromethyl)-1H-pyrazol-4-yl]amino}phenyl)ethyl]carbamoyl}cyclohexane-1-carboxylate

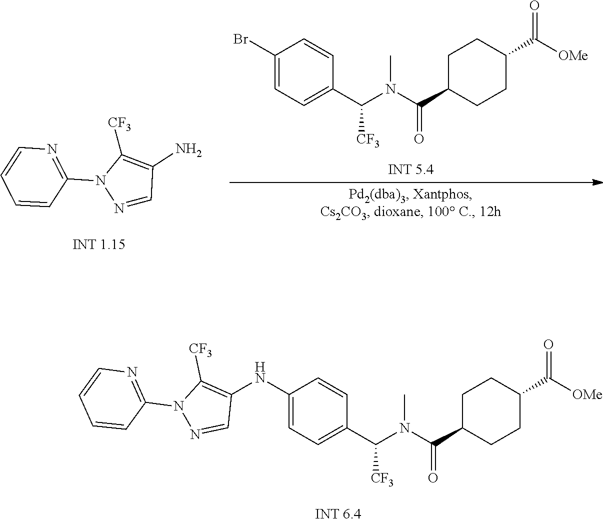 [SMILES: Nc1cnn(-c2ccccn2)c1C(F)(F)F ; COC(=O)[C@H]1CC[C@H](C(=O)N(C)[C@@H](c2ccc(Br)cc2)C(F)(F)F)CC1 ; COC(=O)[C@H]1CC[C@H](C(=O)N(C)[C@@H](c2ccc(Nc3cnn(-c4ccccn4)c3C(F)(F)F)cc2)C(F)(F)F)CC1]

To a solution of 1-(pyridin-2-yl)-5-(trifluoromethyl)-1H-pyrazol-4-amine [INT 1.15](100 mg, 438 μmol), methyl (1r,4r)-4-{[(1S)-1-(4-bromophenyl)-2,2,2-trifluoroethyl](methyl)carbamoyl}cyclohexane-1-carboxylate [INT 5.4](191 mg, 438 μmol), Cs₂CO₃ (426 mg, 1.31 mmol) and xantphos (50.6 mg, 87.6 μmol) in dioxane (3 mL) was added Pd₂(dba)₃ (40.1 mg, 43.8 μmol) and the reaction mixture was stirred at 100° C. for 2 h under N₂. The reaction mixture was concentrated under reduced pressure to give the crude product, which was purified by flash chromatography on silica gel (EtOAc/PE=0/1 to 1/3) to give methyl (1r,4r)-4-{methyl[(1S)-2,2,2-trifluoro-1-(4-{[1-(pyridin-2-yl)-5-(trifluoromethyl)-1H-pyrazol-4-yl]amino}phenyl)ethyl]carbamoyl}cyclohexane-1-carboxylate [INT 6.4](170 mg, 291 μmol, 66.6% yield) as a yellow oil. m/z: [M+H]+ Calcd for C27H28F6N503 584.2; Found 584.1.

Synthesis of MALT1 inhibitors is also described in WO 2023/192506 (PCT/US23/16941) which is incorporated by reference in its entirety for all purposes.

### Pharmaceutical Compositions and Administration

Compounds provided in accordance with the present disclosure are usually administered in the form of pharmaceutical compositions. The disclosure provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. Such compositions may also comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds., which is incorporated by reference in its entirety for all purposes)

Suitable pharmaceutically acceptable excipients are well known to a person skilled in the art. Examples of the pharmaceutically acceptable excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. The pharmaceutically acceptable excipients described in Remington's Pharmaceutical Sciences (Mack Pub. Co., N.J. 1991) (which is incorporated herein by reference in its entirety for all purposes) can be appropriately used. The composition can be formulated into a known form suitable for oral administration, or parenteral administration, for example, injection or infusion. The composition may comprise formulation additives such as a suspending agent, a preservative, a stabilizer and/or a dispersant, and a preservation agent for extending a validity term during storage.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

The pharmaceutical compositions may be administered in either single or multiple doses by any of the accepted modes of administration of agents having similar utilities, for example as described in those patents and patent applications incorporated by reference, including rectal, buccal, sublingual, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery inserted cylindrical polymer.

One mode for administration is parenteral, particularly by injection. The forms in which the novel compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile filtered solution thereof.

Oral administration is another route for administration of compounds in accordance with the disclosure. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragaeanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents: emulsifying and suspending agents; preserving agents such as methyl and propylhydroxy-benzoates: sweetening agents; and flavoring agents.

The compositions of the invention can be formulated so as to provide quick, sustained or delayed release of the active ingredient after administration to the patient by employing procedures known in the art. Controlled release drug delivery' systems for oral administration include osmotic pump systems and dissolution systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, ethyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### MALT1 Associated Diseases

Mucosa-Associated Lymphoid Tissue Lymphoma Translocation Protein 1 (MALT1). MALT1 is a caspase-like protease that plays a role in BCL10-induced activation of NF-κB, and/or activation of mTor pathways (e.g., mTORc1 and/or mTORc2), and/or activation of Jun/Fos. The protein is a component of the CARMA1-BCL1O-MALT1 (CBM) signalosome that triggers NF-κB signaling, and/or mTor pathway signaling (e.g., mTORc1 and/or mTORc2), and/or Jun/Fos signaling to induce lymphocyte activation following antigen-receptor stimulation. A number of MALT1 associated genetic diseases cause constitutive or over-activation activation of NF-κB, and/or over-activation of mTor pathways (e.g., mTORc1 and/or mTORc2), and/or over-activation of Jun/Fos, and/or over-activation of other signaling pathways by increasing activation signals through MALT1 or decreasing down regulation signals through MALT1.

B-cell expansion with NF-κB and T-cell anergy (BENTA), is a rare genetic disorder of the immune system caused by mutations in the gene CARD11 (Caspase recruitment domain family member 11). The disease can be characterized by high levels of certain B-cells starting in infancy (B-cell lymphocytosis), an enlarged spleen (splenomegaly), enlarged lymph nodes (lymphadenopathy), immunodeficiency, and/or an increased risk of lymphoma.

CARD11 encodes a membrane-associated guanylate kinase (MAGUK), a class of proteins that functions as molecular scaffolds for the assembly of multiprotein complexes at specialized regions of the plasma membrane. This protein is also a member of the CARD protein family, which is defined by carrying a characteristic caspase-associated recruitment domain (CARD). The CARD domains of the protein specifically interacts with BCL10, a protein known to function as a positive regulator of cell apoptosis, NF-κB activation, mTor pathways (e.g., mTORc1 and/or mTORc2) activation. When expressed in cells, this protein activated NF-κB, mTor pathways (e.g., mTORc1 and/or mTORc2), and induced the phosphorylation of BCL10.

BENTA disease is inherited in an autosomal dominant manner, or can arise in a de novo fashion from spontaneous mutation of the CARD11 gene. BENTA disease is caused by “gain of-function” mutations in the gene CARD11, which provides instructions for production of the CARD11 protein. These gain-of-function mutations cause the CARD11 protein to be overactive. The CARD11 protein is required for activation of NF-κB and/or mTor pathways (e.g., mTORc1 and/or mTORc2), and/or activation of Jun/Fos in both B and T cells, which is essential for a healthy immune response. The development and differentiation of B cells might also be partially impaired in BENTA disease. BENTA disease is inherited in an autosomal dominant manner. Only one of the two copies of CARD11 needs to be abnormal for a person to have BENTA disease.

In CARD11 gain of function alleles, the MALT1 signaling pathway becomes constitutive, Jun/Fos, and/or NF-κB and/or a mTor pathway (e.g., mTORc1 and/or mTORc2) activation of immune cells occurs constantly. This results in a lymphoproliferative syndrome called BENTA. BENTA patients have elevated levels of inflammation, and are susceptible to recurrent infections, autoimmunity, lymphoma and Hemophagocytic lymphohistiocytosis (HLH). HLH is a rare but potentially fatal condition in which certain white blood cells (histiocytes and lymphocytes) build up in and damage organs, including the bone marrow, liver, and spleen, and destroy other blood cells. BENTA-associated mutations can reside within the N-terminal portion of CARD11 containing the CARD, LATCH and coiled-coil domains. These domains are responsible for CARD11 oligomerization and recruitment of BCL10 and MALT1, making this region a hotspot for CARD11 mutations. CARD11 mutants can spontaneously aggregate to form active signaling clusters with BCL10, MALT1, and active IKK, triggering constitutive NF-κB activation, and/or mTor pathway (e.g., mTORc1 and/or mTORc2) activation, and/or Jun/Fos activation without antigen receptor stimulation. Examples of BENTA-associated mutations include, for example, the following CARD11 mutations R30G, Q, or W, C49Y or S or E or F or N, F1151, T117P, G123S or D, G126R, T128M, F1301 or C, E134G.

BENTA patients often have mild immunodeficiency, and are susceptible to recurrent sinus and lung infections, as well as infections with viruses such as molluscum contagiosum virus, Epstein-Barr virus, or BK virus. BENTA patients are also at increased risk for developing lymphoma (e.g., large B-cell lymphoma).

The majority of B-cells in a BENTA patient's blood are naïve, mature B cells, with elevated levels of a subtype of B cells called transitional B cells. Laboratory studies also have shown poor B-cell differentiation and immunoglobulin, or antibody, secretion. Serum IgM is low in most patients, and total IgG and IgA are typically on the low end of normal. Some patients have poor immune responses to certain vaccines. T-cell counts in people with BENTA disease are within or just above the normal range, although the T cells may be poorly responsive to certain foreign pathogens, hence inclusion of the word “anergy” as part of the BENTA acronym.

Other genetic diseases are linked to increased signaling through the MALT1 pathway. E.g., A20 haploinsufficiency, HOIL1 hypomorphism, CARD14 generalized pustular psoriasis, and NF-kB gain of function syndromes.

A20 haploinsufficiency is an autosomal dominant hereditary disease caused by a pathological mutation in tumor necrosis factor (TNF)-α-induced protein 3 gene. As a result, production of nuclear factor (NF)-κB regulatory protein A20 encoded by TNFAIP3 gene is insufficient. The protein A20, also known as TNAP3, encoded by TNFAIP3, plays a crucial role in the negative regulation of inflammation and immunity.

HOIL1 or HOIP deficiency is associated with an immune disorder involving autoinflammation, immunodeficiency, and inflammatory bowel disease (IBD)-like symptoms. MALT1 paracaspase is a novel negative regulator of LUBAC by proteolytic cleavage of HOIL1. The linear ubiquitin chain assembly complex (LUBAC) which consists of HOIL1, HOIP, and SHARPIN, catalyzes the linear ubiquitination of target proteins-a post-translational modification that is essential for NF-κB activation. HOIL1 or HOIP deficiency is caused by mutations in HOIL1 that cause a loss of function.

Other diseases related to MALT1 include, for example, autoinflammatory conditions (states of severe, recurrent, or persistent inflammation that are seemingly unprovoked), hyperinflammatory conditions (states of states of severe or persistent inflammation that are disproportionately higher than the triggering stimulus), autoimmune or autoinflammatory or hyperinflammatory states that arise due to genetic mutations leading to a state of “gain of function” (including partial gain of function) in the proteins that ordinarily activate or promote activation in a cellular signaling pathway where a protein in that pathway is a target of the MALT1 protease, and autoimmune or autoinflammatory or hyperinflammatory states that arise due to genetic mutations leading to a state of “loss of function” (including partial loss of function) in the proteins that ordinarily inhibit or promote inhibition in a cellular signaling pathway where a protein in that inhibitory pathway is a target of the MALT1 protease.

### Methods of Use

Compounds and compositions described herein are generally useful for modulating MALT1 and are useful for in treating diseases or disorders, in particular those susceptible to modulation of proteolytic and/or autoproteolytic activity of MALT1. In some aspects, the compounds and compositions described herein are useful for inhibiting MALT1. In some aspects, it is contemplated that the compounds and compositions of the present disclosure may be useful in the treatment of a disease, a disorder, or a condition characterized by dysregulated NF-κB, and/or mTor pathway (e.g., mTORc1 and/or mTORc2), and/or Jun/Fos activation, for example, genetic diseases, autoimmune or immunological and inflammatory disorders, allergic disorders, respiratory disorders and oncological disorders. These disease states or conditions, including those above, can be treated by administering the MALT1 inhibitors disclosed herein (e.g., Compounds 1.1, 1.26, 1.27, 1.30, 1.33, 2.1, and/or 2.4) to a patient suffering from the disease, disorder or condition.

Other autoimmune and inflammatory disorders that can be treated with MALT1 inhibitors include, for example, arthritis, ankylosing spondylitis, inflammatory bowel disease, ulcerative colitis, gastritis, pancreatitis, Crohn's disease, celiac disease, multiple sclerosis, systemic lupus erythematosus, lupus nephritis, rheumatoid arthritis, rheumatic fever, gout, organ or transplant rejection, acute or chronic graft-versus-host disease, chronic allograft rejection, Bechet's disease, uveitis, psoriasis, psoriatic arthritis, BENTA disease, A20 haploinsufficiency, HOIL1 or HOIP deficiency, polymyositis, dermatitis, atopic dermatitis, dermatomyositis, acne vulgaris, myasthenia gravis, hidradenitis suppurativa, Grave's disease, Hashimoto thyroiditis, Sjogren's syndrome, and blistering disorders (e.g., pemphigus vulgaris), antibody-mediated vasculitis syndromes, including ANCA-associated vasculitides, Henoch-Schoenlein Purpura, IKBKG gain of function (also called NEMO), IKB gain of function, immune-complex driven diseases (lupus, rheumatoid arthritis, IgA nephritis, IgA vasculitis, IgG vasculitis), and immune-complex vasculitides (either primary or secondary to infection or cancers).

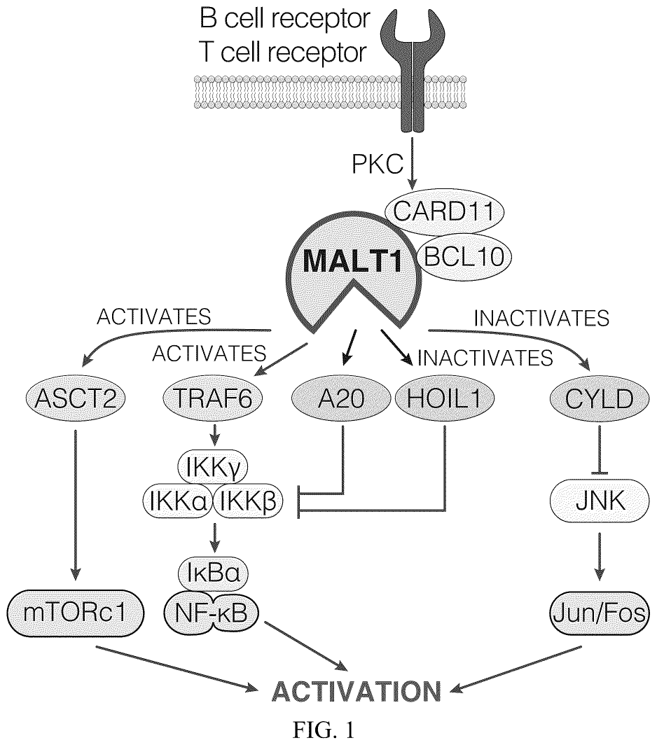

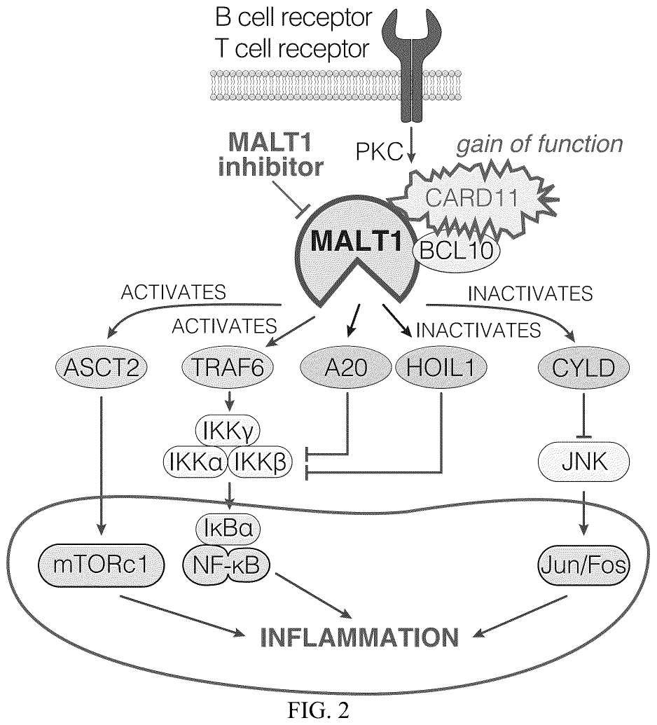

As shown in FIG. 1, MALT1 is a critical component in the signaling pathway for activation of NF-κB, and/or activation of a mTor pathway (e.g., mTORc1 and/or mTORc2), and/or activation of Jun/Fos resulting in activation of immune cells (e.g., B-cells, T-cells, macrophages and/or neutrophils). As shown in FIG. 2, CARD11 mutations can result in gain of function that signals through MALT1 to activate NF-κB, and/or activate a mTor pathway (e.g., mTORc1 and/or mTORc2), and/or activate Jun/Fos to produce BENTA in patients.

The activation of NF-κB by CARD11 alleles that produce gain of function and BENTA can be moderated or prevented using the MALT1 inhibitors (e.g., Compounds 1.1, 1.26, 1.27, 1.30, 1.33, 2.1, and/or 2.4) disclosed herein. Providing the MALT1 inhibitors (e.g., Compounds 1.1, 1.26, 1.27, 1.30, 1.33, and/or 2.4) to a cell (e.g., an immune cell such as B-cells, T-cells, macrophages and/or neutrophils) with activation of NF-κB from CARD11 alleles that produce gain of function and BENTA through the MALT1 signalosome can reduce the activation of NF-κB and reduce the activated state of the cell. In some cases, the MALT1 inhibitor(s) (e.g., Compounds 1.1, 1.26, 1.27, 1.30, 1.33, 2.1, and/or 2.4) can return the cell to a quiescent state.

Patients suffering from CARD11 gain of function mutations (e.g., BENTA) can be treated by administering a MALT1 inhibitor (e.g., Compounds 1.1, 1.26, 1.27, 1.30, 1.33, 2.1, and/or 2.4) disclosed herein. The MALT1 inhibitor (e.g., Compounds 1.1, 1.26, 1.27, 1.30, 1.33, 2.1, and/or 2.4) can be administered in a suitable formulation (e.g., an oral dosage form) at a suitable dose (e.g., 25-1000 mg/day). The MALT1 inhibitor (e.g., Compounds 1.1, 1.26, 1.27, 1.30, 1.33, 2.1, and/or 2.4) produces a therapeutic effect by reversing some or all of the effect of the CARD11 gain of function alleles in the patient (e.g., BENTA patient). CARD11 gain of function mutants can also decrease the killing activity of Natural Killer cells. This effect on NK cells can be reversed by the MALT1 inhibitors disclosed herein for the treatment of viral infections (e.g., herpes family members EBV or CMV). These infections can be quite serious or life-threatening in BENTA patients.

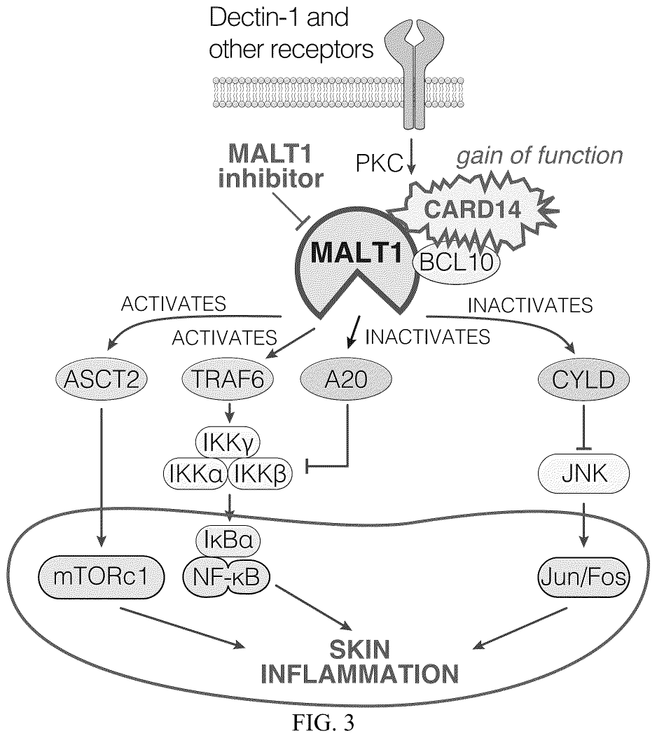

As shown in FIG. 3, CARD14 gain of function mutants in CARD 14 generalized pustular psoriasis interact with MALT1 in a manner analogous to CARD 11 mutants in BENTA.

The activation of NF-κB, and/or a mTor pathway (e.g., mTORc1 and/or mTORc2), and/or Jun/Fos by CARD14 alleles that produce gain of function and CARD14 generalized pustular psoriasis can be moderated or prevented using the MALT1 inhibitors (e.g., Compounds 1.1, 1.26, 1.27, 1.30, 1.33, and/or 2.4) disclosed herein. Providing the MALT1 inhibitors (e.g., Compounds 1.1, 1.26, 1.27, 1.30, 1.33, and/or 2.4) to a cell (e.g., an immune cell such as B-cells, T-cells, macrophages and/or neutrophils) with activation of NF-κB from CARD14 alleles that produce gain of function and CARD14 generalized pustular psoriasis through the MALT1 signalosome can reduce the activation of NF-κB, and/or reduce activation of a mTor pathway (e.g., mTORc1 and/or mTORc2), and/or reduce activation of Jun/Fos thus reducing the activated state of the cell. In some cases, the MALT1 inhibitor(s) (e.g., Compounds 1.1, 1.26, 1.27, 1.30, 1.33, 2.1, and/or 2.4) can return the cell to a quiescent state.

Patients suffering from CARD14 skin inflammation (e.g., generalized pustular psoriasis, pityriasis rubra pilaris) can be treated by administering a MALT1 inhibitor (e.g., Compounds 1.1, 1.26, 1.27, 1.30, 1.33, 2.1, and/or 2.4) disclosed herein. The MALT1 inhibitor (e.g., Compounds 1.1, 1.26, 1.27, 1.30, 1.33, 2.1, and/or 2.4) can be administered in a suitable formulation (e.g., an oral dosage form) at a suitable dose (e.g., 25-1000 mg/day). The MALT1 inhibitor (e.g., Compounds 1.1, 1.26, 1.27, 1.30, 1.33, 2.1, and/or 2.4) produces a therapeutic effect by reversing some or all of the effect of the CARD14 gain of function alleles in the patient suffering from CARD14 generalized pustular psoriasis.

The activation of Jun/Fos, and/or NF-κB and/or a mTor pathway (e.g., mTORc1 and/or mTORc2) by tumor necrosis factor (TNF)-α-induced protein 3 alleles that produce gain of function and A20 haploinsufficiency can be moderated or prevented using the MALT1 inhibitors (e.g., Compounds 1.1, 1.26, 1.27, 1.30, 1.33, 2.1, and/or 2.4) disclosed herein. Providing the MALT1 inhibitors (e.g., Compounds 1.1, 1.26, 1.27, 1.30, 1.33, 2.1, and/or 2.4) to a cell (e.g., an immune cell such as B-cells, T-cells, macrophages and/or neutrophils) with activation of NF-κB and/or a mTor pathway (e.g., mTORc1 and/or mTORc2), and/or Jun/Fos by tumor necrosis factor (TNF)-α-induced protein 3 alleles that produce gain of function and A20 haploinsufficiency through the MALT1 signalosome can reduce the activation of NF-κB and/or a mTor pathway (e.g., mTORc1 and/or mTORc2), and/or Jun/Fos to reduce the activated state of the cell. In some cases, the MALT1 inhibitor(s) (e.g., Compounds 1.1, 1.26, 1.27, 1.30, 1.33, 2.1, and/or 2.4) can return the cell to a quiescent state.

Patients suffering from A20 haploinsufficiency can be treated by administering a MALT1 inhibitor (e.g., Compounds 1.1, 1.26, 1.27, 1.30, 1.33, 2.1, and/or 2.4) disclosed herein. The MALT1 inhibitor (e.g., Compounds 1.1, 1.26, 1.27, 1.30, 1.33, 2.1, and/or 2.4) can be administered in a suitable formulation (e.g., an oral dosage form) at a suitable dose (e.g., 25-1000 mg/day). The MALT1 inhibitor (e.g., Compounds 1.1, 1.26, 1.27, 1.30, 1.33, 2.1, and/or 2.4) produces a therapeutic effect by reversing some or all of the effect of the tumor necrosis factor (TNF)-α-induced protein 3 gain of function alleles in the patient suffering from A20 haploinsufficiency.

The activation of Jun/Fos, and/or NF-κB and/or a mTor pathway (e.g., mTORc1 and/or mTORc2) by HOIL1 alleles that produce loss of function and HOIL1 or HOIP deficiency can be moderated or prevented using the MALT1 inhibitors (e.g., Compounds 1.1, 1.26, 1.27, 1.30, 1.33, 2.1, and/or 2.4) disclosed herein. Providing the MALT1 inhibitors (e.g., Compounds 1.1, 1.26, 1.27, 1.30, 1.33, 2.1, and/or 2.4) to a cell (e.g., an immune cell such as B-cells, T-cells, macrophages and/or neutrophils) with activation of NF-κB from CARD14 alleles that produce gain of function and HOIL1 or HOIP deficiency through the MALT1 signalosome can reduce the activation of NF-κB and reduce the activated state of the cell. In some cases, the MALT1 inhibitor(s) (e.g., Compounds 1.1, 1.26, 1.27, 1.30, 1.33, 2.1, and/or 2.4) can return the cell to a quiescent state.

In general, undesired activation of Jun/Fos and/or NF-κB and/or mTor a pathway (e.g., mTORc1 and/or mTORc2) through the MALT1 signalosome from any source (e.g., genetic and/or receptor stimulation) can be moderated or prevented using the MALT1 inhibitors (e.g., Compounds 1.1, 1.26, 1.27, 1.30, 1.33, 2.1, and/or 2.4) disclosed herein. Providing the MALT1 inhibitors (e.g., Compounds 1.1, 1.26, 1.27, 1.30, 1.33, 2.1, and/or 2.4) to a cell (e.g., an immune cell such as B-cells, T-cells, macrophages and/or neutrophils) with undesired activation of Jun/Fos, and/or NF-κB and/or a mTor pathway (e.g., mTORc1 and/or mTORc2) through the MALT1 signalosome can reduce the activation of NF-κB and reduce the activated state of the cell. In some cases, the MALT1 inhibitor(s) (e.g., Compounds 1.1, 1.26, 1.27, 1.30, 1.33, 2.1, and/or 2.4) can return the cell to a quiescent state.

A compound of composition described herein may be administered in combination with another agent or therapy. A subject to be administered a compound disclosed herein may have a disease, disorder, or condition, or a symptom thereof, that would benefit from treatment with another agent or therapy. The compound of composition described herein may be administered either simultaneously with, or before or after, one or more other therapeutic agent. The compound of composition described herein may be administered separately, by the same or different route of administration, or together in the same pharmaceutical composition as the other agents. The compound described herein may be administered as the sole active ingredient or in conjunction with, e.g., as an adjuvant to, other drugs e.g., immunosuppressive or immunomodulating agents or other anti-inflammatory agents, e.g., for the treatment or prevention of allo- or xenograft acute or chronic rejection or inflammatory or autoimmune disorders, or a chemotherapeutic agent, e.g., a malignant cell anti-proliferative agent. For example, the compounds of the invention may be used in combination with a calcineurin inhibitor, e.g., cyclosporin A or FK 506; a mTOR inhibitor, e.g., rapamycin, 40-0-(2-hydroxyethyi)-rapamycin, biolimus-7 or biolimus-9; an ascomycin having immunosuppressive properties, e.g., ABT-281, ASM981; corticosteroids; cyclophosphamide; azathioprine; methotrexate; leflunomi.de; mizoribine; mycophenolic acid or salt; mycophenolate mofetil; or IL-1 beta inhibitor.

Suitable genetic tests for CARD11 gain of function mutants (alleles) that produce BENTA can be performed using well known methods. Such methods include, for example, high throughput genome sequencing, exome sequencing, FISH techniques, microarrays, other hybridization technologies, PCR related diagnostics, other nucleic acid amplification technologies, CRISPR diagnostics, denaturing HPLC, gene expression profiling, pharmacokinetic testing of CARD11, and/or metabolite levels related to CARD11/MALT1/NF-κB.

Various features and embodiments of the disclosure are illustrated in the following representative examples, which are intended to be illustrative, and not limiting. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: MALT1 Inhibitors Reverse the Gain of Function by CARD11 Variants

MALT1 inhibitors (e.g., Compounds 1.1, 1.26, 1.27, 1.30, 1.33, 2.1, and/or 2.4) are incubated with T cells that lack endogenous CARD11 and bear transiently transfected CARD11 cDNA bearing gain-of-function mutations. An optional variation is co-transfecting 50:50 a copy of the wildtype CARD11 gene with the CARD11 gain of function mutant. The T-cells include a reporter gene (κB-GFP) that acts as a readout of NFκB signaling. The expression of NF-κB-GFP at baseline and after anti-CD3, or PMA/ionomycin stimulation is measured.

T-Cells bearing GOF variants of CARD11 have elevated baseline activation of NF-κB-GFP, and higher still after stimulation, which is assessed by flow cytometry. The MALT1 inhibitors (e.g., Compounds 1.1, 1.26, 1.27, 1.30, 1.33, 2.1, and/or 2.4) suppress the effect of the CARD11 gain of function mutants resulting in lowered NF-κB-GFP expression.

### Example 2: Clinical Trial of MALT1 Inhibitors for BENTA

MALT1 inhibitors (e.g., Compounds 1.1, 1.26, 1.27, 1.30, 1.33, 2.1, and/or 2.4) are used to treat patients with BENTA. MALT1 inhibitors (e.g., Compounds 1.1, 1.26, 1.27, 1.30, 1.33, 2.1, and/or 2.4) are formulated in an oral dosage form and administered to patients once daily at a starting dose of 25 mg/day. Alternatively, the daily dose is escalated from 25 mg/day to 1000 mg/day.

Patients receiving the MALT1 inhibitors (e.g., Compounds 1.1, 1.26, 1.27, 1.30, 1.33, 2.1, and/or 2.4) show reduced Jun/Fos, and/or NF-κB and/or a mTor pathway (e.g., mTORc1 and/or mTORc2) activation and amelioration of their BENTA symptoms.

### Example 3: MALT1 Inhibitors Reverse a mTor Pathway (e.g., mTORc1 and/or mTORc2) Activation by Gain of Function CARD11 Variants

Primary T cells are stimulated to activation and cultured in the presence of IL-2. For stimulation, cells are preincubated with anti-CD3F and anti-CD28 on ice and washed. Cells were stimulated by crosslinking the anti-CD3 antibody with a secondary antibody (e.g., anti-mouse IgG for 10 min) in complete T cell media and then fixed with pre-warmed fixation buffer. Cells are then washed with FACS buffer and permeabilized with permeabilization buffer pre-chilled to −20° C. and incubated for 30 min on ice. Cells are washed and then stained with the following antibodies or appropriate isotype controls anti-pAkt (Ser473) and anti-pS6 (Ser240/244).

MALT1 inhibitors (e.g., Compounds 1.1, 1.26, 1.27, 1.30, 1.33, 2.1, and/or 2.4) are incubated with the primary T cells after activation. The MALT1 inhibitors (e.g., Compounds 1.1, 1.26, 1.27, 1.30, 1.33, 2.1, and/or 2.4) suppress mTor pathway (e.g., mTORc1 and/or mTORc2) activation.

### Example 4: MALT1 Inhibitors Reverse the Gain of Function by CARD14 Variants

MALT1 inhibitors (e.g., Compounds 1.1, 1.26, 1.27, 1.30, 1.33, 2.1, and/or 2.4) are incubated with epithelial cells (e.g., HEK293 cells) that lack endogenous CARD14 and bear transiently transfected CARD14 cDNA bearing gain-of-function mutations. The epithelial cells include a reporter gene (κB-GFP) that acts as a readout of NFκB signaling. The expression of NF-κB-GFP at baseline and after anti-CD3, or PMA/ionomycin stimulation is measured.

Epithelial cells bearing gain of function variants of CARD14 have elevated baseline activation of NF-κB-GFP, and higher still after stimulation, which is assessed by flow cytometry. The MALT1 inhibitors (e.g., Compounds 1.1, 1.26, 1.27, 1.30, 1.33, 2.1, and/or 2.4) suppress the effect of the CARD14 gain of function mutants resulting in lowered NF-κB-GFP expression.

### Example 5: MALT1 Cleavage of HOIL1 is Reduced by MALT1 Inhibitors

Recombinant full-length human MALT1 protein was expressed and purified. C-terminal Myc-FLAG-tagged full-length human HOIL1 was obtained from Origene. HOIL1 protein (0.05 μg/μL) is incubated with different concentrations of MALT1 in assay buffer (200 mM Tris-HCl, 0.8 M Na citrate, 0.1 mM EGTA, 0.05% CHAPS, 1 mM DTT, pH 7.4) for 2 h at 37° C. Cleavage of HOIL1 was analyzed by gel electrophoresis using a 4-12% Bis-Tris SDS-polyacrylamide (PAGE) gradient gel (Life Technologies). The presence of a small fragment was confirmed by immunoblotting with an N-terminal antibody (anti-N-terminal HOIL1; HPA024185; Sigma). Total HOIL1 was observed using a C-terminal directed antibody (anti-FLAG, clone M2, Sigma). These assays are performed with both the cleavable HOIL1 and, as a negative control, a non-cleavable HOIL1 (where Lys is substituted for Arg165).

MALT1 inhibitors (e.g., Compounds 1.1, 1.26, 1.27, 1.30, 1.33, 2.1, and/or 2.4) are added to the reaction mix for HOIL1 cleavage, and cleavage of HOIL1 is reduced.

### Example 6: MALT1 Inhibitors Reduce NF-κB Activity In CARD11 Gain of Function Mutants in Jurkat Cells

Jurkat cells were engineered with the E134G, C49Y, or G123D CARD11 gain of function (GOF) mutant. The Jurkat cells included a reporter gene (κB-GFP) that acts as a readout of NFκB signaling.

Jurkat cells bearing GOF E134G, C49Y, or G123D CARD11 had elevated baseline activation of NF-κB-GFP. MALT1 inhibitors (e.g., Compound 1.1 or 2.4) were incubated with the above engineered Jurkat cells. The MALT1 inhibitors (e.g., Compounds 1.1 or 2.4) suppress the effect of the CARD11 gain of function mutants resulting in lowered NF-κB-GFP expression for all three GOF CARD11 mutants. These MALT1 inhibitors reduced NF-κB-GFP expression in a dose dependent fashion from about 100 nM to 5 μM for both Compound 1.1 or compound 2.4.

Jurkat cells bearing GOF E134G, C49Y, or G123D CARD11 were also assessed for cleavage of CYLD as a measure of the proteolytic function of MALT1. In the absence of inhibitors, the Jurkat cells with the GOF CARD11 mutants cleaved CYLD. The MALT1 inhibitors (e.g., Compounds 1.1 or 2.4) suppress this CYLD cleavage in a dose dependent manner in the range of about 100 nM to 10 μM.

### Example 7: MALT1 Inhibitors Reduce NF-κB Activity in CARD11 Gain of Function Mutants in B-Cells

B-cells are obtained from patients with BENTA (B-cell Expansion with NF-κB and T-cell Anergy). Both resting and activated (CD40L+IL-4) B cells are treated with MALT1 inhibitors (e.g., Compounds 1.1, 1.26, 1.27, 1.30, 1.33, and/or 2.4).

Changes in steady-state CARD11-induced NF-κB signaling are measured at various timepoints over ~24 hrs by monitoring CARD11 aggregation and nuclear accumulation of p65 or direct quantification of active NF-κB complexes in nuclear lysates (TransAm assays). MALT1 substrate cleavage will also be monitored over time.

Changes in the expression of NF-κB-dependent genes known to be elevated in BENTA B cells (e.g. BCL2, NFκB2, c-FLIP, cyclin D1) are assessed by quantitative PCR and/or immunoblotting of whole cell lysates −/+MALT1 inhibitors (e.g., Compounds 1.1, 1.26, 1.27, 1.30, 1.33, and/or 2.4). Alternatively, bulk RNA-seq can be performed on BENTA B cells −/+MALT1 inhibitors (e.g., Compounds 1.1, 1.26, 1.27, 1.30, 1.33, and/or 2.4) to assess changes to the entire B cell transcriptome.

CARD11 gain of function is known to promote survival and proliferation of B-cells, which can result in excessive numbers of B cells like in BENTA or B cell lymphomas. MALT1 inhibitors reduce this survival. Survival and proliferation of healthy or BENTA B-cells are assessed in response to combinations of stimuli (e.g., anti-IgM/IgD, rCD40L/IL-4/IL-21, pokeweed mitogen, etc.) −/+MALT1 inhibitors (e.g., Compounds 1.1, 1.26, 1.27, 1.30, 1.33, and/or 2.4).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

1. A method for reducing activation of a NF-κB caused by a gain of function allele that acts on a MALT1, comprising the steps of: obtaining a cell with the gain of function allele; and exposing the cell to a MALT1 inhibitor whereby the activation of NF-κB is reduced.

2. A method for treating a patient with a CARD11 gain of function mutation, comprising the steps of: obtaining the patient with BENTA; and administering to the patient to a therapeutically effective amount of a MALT1 inhibitor whereby the activation of a NF-B in the patient is reduced.

3. The method of claim 1, wherein the cell is BENTA cell.

4. The method of claim 1, wherein the gain of function allele is a CARD14 gain of function allele.

5. (canceled)

6. The method of claim 4, wherein the cell with the CARD14 gain of function allele is from a pustular psoriasis cell or a pityriasis rubra pitaris cell.

7. (canceled)

8. (canceled)

9. (canceled)

10. The method of claim 1, wherein the gain of function allele is a CARD11 gain of function allele.

11. (canceled)

12. The method of claim 1, wherein the cell is an immune cell.

13. The method of claim 12, wherein the immune cell is a B-cell, a T-cell, a macrophage, or a neutrophil.

14. The method of claim 13, wherein the immune cell is a B-cell.

15. The method claim 1, wherein the MALT1 inhibitor is a compound of Formula 1, 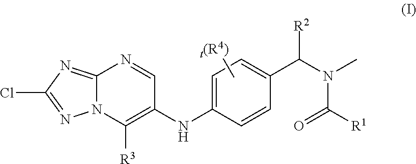
or a pharmaceutically acceptable salt thereof, wherein:
R1 is selected from the group consisting of C1-6 alkyl, C1-6 alkoxy, C3-6 cycloalkyl and 5-10 membered heterocyclyl, wherein the C1-6 alkyl, C3-6 cycloalkyl, and 5-10 membered heterocyclyl may be optionally substituted on one or more available carbons by one, two, three, or more substituents each independently selected from R1a, wherein if the 5-10 membered heterocyclyl contains a substitutable ring nitrogen atom, that ring nitrogen atom may optionally be substituted by R1b, and wherein if the 5-10 membered heterocyclyl contains a substitutable ring sulfur atom, that ring sulfur atom may be optionally substituted with two O atoms;
R2 is CH3 or CF3;
R3 is hydrogen; or
R3 is selected from the group consisting of C1-6 alkyl, C1-6 alkoxy, C3-7 cycloalkyl, 5-6 membered heterocyclyl, 5-6 membered heterocyclyl-C1-3 alkyl 5-6 membered heterocyclyl-O—, phenyl, and 5-6 membered heteroaryl, any of which may be optionally substituted with one, two or three substituents each independently selected from R3a;
R4 is C1-6 alkyl;
R1a is independently, for each occurrence, selected from the group consisting of cyano, halogen, hydroxyl, oxo, C1-6 alkyl, —C(O)ORA, —C(O)N(RA)₂, —N(RA)₂, C1-6 alkoxy, 5-6 membered heterocyclyl, and 5-6 membered heteroaryl, wherein the C1-6 alkyl is optionally substituted with N(RA)₂, and wherein if the 5-6 membered heterocyclyl contains a substitutable ring nitrogen atom, that ring nitrogen atom may optionally be substituted by R^{p};
R1b is selected from the group consisting of C1-6 alkyl, —C(O)ORA, —C(O)C1-6 alkyl, —C(O)C3-6 cycloalkyl, —C(O)N(RA)₂, and —S(O)₂C1-6 alkyl;
R3a is independently, for each occurrence, selected from the group consisting of halogen, C1-4 alkyl, C1-4 haloalkyl, C1-4 alkoxy, C1-4 haloalkoxy, hydroxy, C1-4 alkenyl, cyano, azido, —NRCRD, C3-6 cycloalkyl, Ci1-4 alkoxy, 5-6 membered heterocyclyl-O—, 5-6 membered heterocyclyl, and phenyl, wherein C3-6cycloalkyl, 5-6 membered heterocyclyl-O—, 5-6 membered heterocyclyl, and phenyl are optionally substituted with one, two or three substituents each independently selected from Rp;
Rp is independently, for each occurrence, selected from the group consisting of halogen, C1-4 alkyl, C1-4 haloalkyl, hydroxy, C1-4 alkoxy, C1-4 alkoxyC1-4 alkyl, NRCRD, and aminoC1-3 alkyl;
RA is independently, for each occurrence, selected from the group consisting of hydrogen, C1-6 alkyl, —C(O)C1-6 alkyl, and —C(O)OC1-6 alkyl;
RB is selected from the group consisting of Ci1-6 alkyl, C1-6 cycloalkyl, and —C(O)OC1-6 alkyl;
RC and RD are independently, for each occurrence, selected from the group consisting of hydrogen, C1-6 alkyl, haloC1-6 alkyl, and C-3-4 cycloalkyl, or
RC and RD together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and
T is 0 or 1.

16. The method of claim 15, wherein the MALT1 inhibitor comprises a compound 1.1, a compound 1.26, a compound 1.27, a compound 1.30, or a compound 1.33, or a combination of the foregoing.

17. The method of claim 16, wherein the MALT1 inhibitor comprises a compound 1.1.

18. The method of claim 16, wherein the MALT1 inhibitor comprises a compound 1.26.

19. The method of claim 16, wherein the MALT1 inhibitor comprises a compound 1.27.

20. The method of claim 16, wherein the MALT1 inhibitor comprises a compound 1.30.

21. The method of claim 16, wherein the MALT1 inhibitor comprises a compound 1.33.

22. The method of claim 1, wherein the MALT1 inhibitor is a compound of Formula Ia, 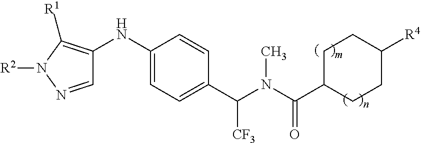
or a stereoisomer and/or a pharmaceutically acceptable salt thereof, wherein:
R1 is C1-6alkyl or C1-3haloalkyl, wherein the C1-6alkyl may be optionally substituted with —O—C1-3alkyl;
R2 is aryl or 5-6 membered heteroaryl, wherein the aryl may be optionally substituted with cyano;
R4 is —C(O)OH or 5-6 membered heteroaryl;
m is 0 or 1; and
n is 0 or 1.

23. The method of claim 22, wherein the MALT1 inhibitor is compound 2.1 or compound 2.4.

24. The method of claim 23, wherein the MALT1 inhibitor is compound 2.1.

25. The method of claim 23, wherein the MALT1 inhibitor is compound 2.4.
